(19) United States
(12) Patent Application Publication
(10) Pub. No.: US 2022/0062190 A1
(43) Pub. Date: Mar. 3, 2022

(54) Therapeutic Agent and Nutraceutical Compositions And Methods for Making and Using Same

(71) Applicant: NUREVELATION, LLC, Durham, NC (US)

(72) Inventors: Tolan Naomie Lucas, Raleigh, NC (US); Eric Seidel, Durham, NC (US); Mark Anthony Bernard, Jersey City, NJ (US); Robin Lee Geller, Buffalo Grove, IL (US); César Andrés Carrasco-López, Lawrence Township, NJ (US); Davide Deodato, Abu Dhabi (AE)

(21) Appl. No.: 17/459,728

(22) Filed: Aug. 27, 2021

Related U.S. Application Data
(60) Provisional application No. 63/071,820, filed on Aug. 28, 2020.
(60) Provisional application No. 63/232,006, filed on Aug. 11, 2021.

(51) Int. Cl. A61K 9/51; A61K 47/26; A23L 33/125

(52) U.S. Cl. CPC A61K 9/5123; A23V 2002/00; A23L 33/125; A61K 47/26

(57) ABSTRACT

Provided are therapeutic agent or nutraceutical compositions recovered from a dispersion comprising a sugar, sugar alcohol, or sugar substitute; water; and a therapeutic agent or nutraceutical. Also provided are products comprising the therapeutic agent or nutraceutical compositions, including a variety of therapeutic products, as well as methods of using the compositions.

### CROSS-REFERENCE TO RELATED APPLICATIONS

This application claims the benefit of U.S. Provisional Application No. 63/071,820, filed Aug. 28, 2020, and U.S. Provisional Application No. 63/232,006, filed Aug. 11, 2021, both of which are incorporated by reference in their entirety.

### BACKGROUND

The nature of various therapeutic agents and nutraceutical compositions including their solubility and polarity make it challenging to incorporate such agents and nutraceuticals into carrier complexes and syrups suitable for administration to a subject such as a human or animal. Additionally, existing methods for delivering therapeutic agents and nutraceuticals to subjects can be impaired by poor absorption and in some cases problems associated with the inability of the therapeutic agent to cross the blood-brain barrier. Accordingly, a need in the art exists for a method for incorporating a variety of therapeutic and nutraceutical agents into suitable compositions. These needs and others are met by the following methods and products.

### SUMMARY

In one aspect, this disclosure relates to a method of incorporating a therapeutic agent or nutraceutical into a composition in which the therapeutic agent or nutraceutical is physically aggregated or noncovalently associated with a sugar, sugar alcohol, or sugar substitute (and in some aspects encapsulated by the sugar, sugar alcohol, or sugar substitute in the form of a nanoparticle composition), thereby improving properties of the therapeutic agent or nutraceutical composition such as bioavailability and absorption, among others. The methods also result in compositions that have improved solubility, thus eliminating problems associated with handling during manufacturing. Additionally, the methods result in altered and in some instances improves routes of administering a therapeutic agent or nutraceutical.

In a specific aspect, disclosed is a method for making a therapeutic agent or nutraceutical composition, the method comprising forming a dispersion comprising a sugar, sugar alcohol, or sugar substitute; water; and an effective amount of the therapeutic agent or nutraceutical; wherein the mass ratio of the therapeutic agent or nutraceutical to the sugar, sugar alcohol, or sugar substitute is from about 1:50 to 1:10; and the mass ratio of the sugar, sugar alcohol, or sugar substitute to the water is from about 1:8 to about 1:1.5; heating the dispersion at a temperature equivalent to from 90° C. to 122° C. at a pressure of 1 atm for a sufficient time to reduce the volume of the dispersion; and after the heating step, recovering the therapeutic agent or nutraceutical composition from the dispersion.

In some aspects, the therapeutic agent or nutraceutical composition recovered from the dispersion is in a form selected from: a solidified complex, or syrup, characterized by a physical aggregation or noncovalent chemical association of the therapeutic agent or nutraceutical and the sugar, sugar alcohol, or sugar substitute; wherein the syrup, when present, has a viscosity ranging from about 15 cp to about 25,000 cp; or nanoparticles comprising the therapeutic agent or nutraceutical encapsulated by the sugar, sugar alcohol, or sugar substitute, the nanoparticles having a particle size ranging from 200 nm to 2,000 nm.

In one aspect, the therapeutic agent, when present, has a molecular weight of 120 g/mol to 6,000 g/mol; and when the therapeutic agent is a solid at a temperature equivalent to 25° C. at a pressure of 1 atm, the therapeutic agent has a melting point of 100° C. to 390° C.; and when the therapeutic agent is a liquid at a temperature equivalent to 25° C. at a pressure of 1 atm, the therapeutic agent has a boiling point of at least 130° C.

In a further aspect, disclosed is a therapeutic agent or nutraceutical composition consisting essentially of a sugar, sugar alcohol, or sugar substitute; an effective amount of the therapeutic agent or nutraceutical; wherein when the therapeutic agent or nutraceutical composition is in the form of a solid complex or nanoparticles, the therapeutic agent or nutraceutical composition comprises less than 25% water by weight of the composition; and when the therapeutic agent or nutraceutical composition is in the form of a syrup, the therapeutic agent or nutraceutical composition comprises 30% to 60% water by weight of the composition; wherein the therapeutic agent, when present, has a molecular weight of 120 g/mol to 6,000 g/mol; and when the therapeutic agent is a solid at a temperature equivalent to 25° C. at a pressure of 1 atm, the therapeutic agent has a melting point of 100° C. to 390° C.; and when the therapeutic agent is a liquid at a temperature equivalent to 25° C. at a pressure of 1 atm, the therapeutic agent has a boiling point of at least 130° C.

In a further aspect, the disclosure relates to a product comprising the therapeutic agent or nutraceutical composition prepared by a disclosed method.

In an additional aspect, disclosed is a method of treating a condition in a subject, comprising administering to the subject the therapeutic agent or nutraceutical composition prepared by a disclosed method. In a further aspect, disclosed is a method of treating a condition in a subject, comprising administering to the subject a disclosed therapeutic agent or nutraceutical composition.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which is incorporated in and constitutes part of this specification and together with the description, serves to explain the principles of the disclosure.

FIG. 1 is a process flow diagram illustrating an exemplary aspect of making a disclosed solidified therapeutic agent or nutraceutical composition, which can be in the form of a solid complex or nanoparticle.

FIG. 2 is a process flow diagram illustrating an exemplary aspect of making a disclosed therapeutic agent or nutraceutical syrup.

FIG. 3 is a transmission electron microscopy (TEM) image of an exemplary nanoparticle-encapsulated hydrochlorothiazide sample prepared according to the disclosed methods.

FIG. 4A is a reference mass spectrum of fenofibrate which shows the fenofibrate mass peak at 361.

FIG. 4B shows a mass spectrum of an exemplary nanoparticle-encapsulated fenofibrate, showing the fenofibrate mass peak at 361. The mass spectrum shown in FIG. 4B demonstrates that the fenofibrate remains stable, i.e., does not decompose, when encapsulated according to the disclosed methods.

FIG. 5A is a reference NMR spectrum of pyrazinamide.

FIG. 5B shows an NMR spectrum of an exemplary nanoparticle-encapsulated pyrazinamide, which shows the pyrazinamide peaks as well as signals in the 2.5-5.5. region corresponding to sucrose.

### DETAILED DESCRIPTION

The present invention can be understood more readily by reference to the following detailed description of the invention and the Examples included therein.

Disclosed are components that can be used to perform the disclosed methods. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and products. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

While aspects of this disclosure can be described and claimed in a particular statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present application is not entitled to antedate such publication by virtue of prior invention. Further, stated publication dates may be different from actual publication dates, which can require independent confirmation.

### A. Definitions

Listed below are definitions of various terms. These definitions apply to the terms as they are used throughout this specification, unless otherwise limited in specific instances, either individually or as part of a larger group.

As used in the specification and in the claims, the term “comprising” can include the aspects “consisting of” and “consisting essentially of”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

References in the specification and concluding claims to parts by weight of a particular element or component in a composition or product, denotes the weight relationship between the element or component and any other elements or components in the composition or product for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

As used herein, the term “sugar” is a collective term encompassing a variety of monosaccharides (e.g., glucose, dextrose, fructose, galactose), disaccharides (e.g., sucrose, lactose, maltose, trehalose), and oligosaccharides or polysaccharides (e.g., maltodextrin).

The term “sugar alcohol,” as used herein, refers to organic compounds, typically derived from a sugar, containing one hydroxyl group attached to each carbon atom. Sugar alcohols are also known as polyhydric alcohols, polyalcohols, alditols or glycitols.

As used herein, the term “sugar substitute” refers to a food additive that provides a sweet taste similar to that of sugar while containing less food energy than sugar-based sweeteners, e.g., a zero-calorie or low-calorie sugar substitute. Suitable sugar substitutes include those that do not include sucrose, fructose, or glucose, for example. The sugar substitute can be natural (e.g., plant derived) or artificial.

“Stevia,” as used herein, refers to any product derived from Stevia rebaudianatype including the leaves thereof, and any product comprising steviol glycosides. Examples include without limitation liquid or solid stevia sweeteners, e.g., stevia extract, stevia leaf extract, stevia powder, stevia extract powder, organic stevia, sugar-free stevia, and the like.

“Disperson” refers to a divided solid or in some aspects liquid in a continuous liquid medium. In one aspect, the “dispersion” is a mixture.

“Complex,” as used herein, refers to a solid entity formed by physical aggregation or noncovalent chemical association (e.g., through one or more noncovalent bonds) of two or more molecular entities, e.g., a therapeutic agent or nutraceutical and a sugar, sugar alcohol, or sugar substitute. “Therapeutic agent or nutraceutical complex,” as used herein, refers to a solid complex of a therapeutic agent or nutraceutical and a sugar, sugar alcohol, or sugar substitute, and includes for example, complexes in which the therapeutic agent or nutraceutical is at least partially encapsulated by the sugar, sugar alcohol, or sugar substitute. “Therapeutic agent or nutraceutical complex” also includes complexes in which the therapeutic agent or nutraceutical and sugar, sugar alcohol, or sugar substitute is self-assembled through physical aggregation or noncovalent chemical association, in addition to complexes that have a micelle or micelle-like structure. In some aspects, physical aggregation or noncovalent chemical association can be determined by a suitable method such as transmission electron microscopy (TEM).

“Nanoparticle,” as used herein, refers to a solid nanoparticle entity formed by physical aggregation or noncovalent chemical association (e.g., through one or more noncovalent bonds) of two or more molecular entities, e.g., a therapeutic agent or nutraceutical and a sugar, sugar alcohol, or sugar substitute. In one aspect, a disclosed therapeutic agent or nutraceutical is encapsulated within the nanoparticle. “Therapeutic agent or nutraceutical nanoparticle,” as used herein, refers to a solid nanoparticle encapsulating a therapeutic agent or nutraceutical with a sugar, sugar alcohol, or sugar substitute carrier, and includes for example, nanoparticles in which the therapeutic agent or nutraceutical is at least partially encapsulated by the sugar, sugar alcohol, or sugar substitute. “Therapeutic agent or nutraceutical nanoparticle” also includes nanoparticles in which the therapeutic agent or nutraceutical and sugar, sugar alcohol, or sugar substitute is self-assembled through physical aggregation or noncovalent chemical association, in addition to nanoparticles that have a micelle or micelle-like structure. In general, the disclosed nanoparticles have a size ranging from about 200 nm to about 2,000 nm, e.g., from about 200 nm to about 1,000 nm, or from about 200 nm to about 500 nm. In one aspect, the nanoparticles can be present as an aggregate. The aggregate can have a size ranging from about 200nm to about 2,000 nm, e.g., from about 200 nm to about 1,000 nm, or from about 200 nm to about 500 nm. Particle size can be determined using methods known in the art, e.g., light scattering, zeta potential measurements, or TEM. Particle size, as referred to herein, refers to the mean or average particle size of a given therapeutic agent or nutraceutical nanoparticle sample.

“Syrup,” as used herein, refers to a solution or mixture having a viscosity ranging from about 15 centipoise (cp) to about 25,000 cp. “Therapeutic agent or nutraceutical syrup,” as used herein, refers to a solution or mixture comprising a therapeutic agent or nutraceutical together with a sugar, sugar alcohol, or sugar substitute, having a viscosity ranging from about 15 cp to about 25,000 cp. The therapeutic agent or nutraceutical and the sugar, sugar alcohol, or sugar substitute in the syrup can be associated by physical aggregation or noncovalent chemical association (e.g., through one or more noncovalent bonds). For example, the therapeutic agent or nutraceutical can be encapsulated by the sugar, sugar alcohol, or sugar substitute, or the therapeutic agent or nutraceutical and sugar, sugar alcohol, or sugar substitute can be self-assembled through physical aggregation or noncovalent chemical association, in addition to having a micelle or micelle-like structure.

The term “mass ratio,” as used herein, refers to the mass of one substance (S1) relative to the mass of another substance (S2), where both masses have identical units (e.g., grams), expressed as S1:S2. For a substance such as water with a density of about 1 mg/mL, it is understood that reference to a volume of water (e.g., in mL) is equivalent to mass (e.g., in units of mg).

The terms “fruit water” and “plant water,” as used herein, refer to the liquid fluid or juice that can be derived from the fruit, plant, or a vegetable produced by the plant. Non-limiting examples of fruit waters include coconut water, pineapple water, cherry water, mango water, apple water, pomegranate water, and the like. Non-limiting examples of plant or vegetable waters include cactus water, aloe vera water, beet water, carrot water, and the like. The liquid or juice derived from the fruit, plant, or vegetable produced by the plant is independent of the method of production and can be obtained by extraction, blending, infusion, and other methods known in the art.

A temperature or temperature range, as expressed herein, refers to the temperature or temperature range at a pressure of 1 atm and equivalents thereof. For example, the phrase “at a temperature equivalent to from about 90° C. to about 122° C. at a pressure of 1 atm” refers not only to the temperature range at the stated atmospheric pressure but also to equivalent temperatures at lower and higher atmospheric pressures. Thus, a stated temperature range can encompass a lower equivalent temperature range at a pressure lower than 1 atm and a higher equivalent temperature range at a pressure higher than 1 atm. Similarly, in some aspects, a stated temperature range can encompass a higher equivalent temperature range at a pressure lower than 1 atm to a achieve a kinetic energy equivalent to that achieved at the stated temperature range.

Equivalent boiling temperatures at different pressures can be calculated according to the Clausius-Clapeyron equation:

$$ln ( {P}_{2} ) - ln ( {P}_{1} ) = \frac{Δ {H}_{vap}}{R} [ \frac{1}{{T}_{1}} - \frac{1}{{T}_{2}} ]$$

where P₁ and T₁ are standard atmospheric pressure and the known boiling point of water, respectively, ΔHᵥₐₚ is the enthalpy of vaporization of water, and R is the gas constant (8.3145 J/mol*K). Using the Clausius-Clapeyron equation, for example, it can be determined that the boiling temperature of water at 2 atm of pressure (about 119° C.) can be equivalent to the boiling temperature of water at 1 atm of pressure (about 100° C.). Other known methods for determining temperatures equivalent to stated temperatures at stated atmospheric pressures can also be used, e.g., according to the phase diagram of the solvent used such as water.

“LogP,” as used herein, refers to the logarithm of the ratio of equilibrium concentrations of unionized therapeutic agent between octanol and water. The “octanol-water partition coefficient” can quantify the hydrophobicity or lipophilicity of a given therapeutic agent.

“pKa,” as used herein, refers to the negative logarithmic of the dissociation constant Kₐ for an acid or conjugate acid in a given solvent: pKₐ=−Logi₀Kₐ. Kₐ, also known as the acidity constant, can be determined from the following:

$${K}_{a} = \frac{[ A - ] [ SH + ]}{[ HA ] [ S ]}$$

for the reaction: HA+S [symbol] A⁻+SH⁺, where S is the solvent and HA is an acid that dissociates into A⁻, known as the conjugate base of the acid, and a hydrogen ion which combines with a solvent molecule. When the concentration of solvent molecules is constant, Kₐ is defined as

$${K}_{a} = \frac{[ A - ] [ H + ]}{[ HA ]}$$

The term “cannabinoid,” as used herein, refers to a class of chemical compounds capable of interacting with any mammalian cannabinoid receptor, for example the human CB₁ or CB₂ receptor. The term encompasses naturally-occurring cannabinoids (e.g., phytocannabinoids found in the cannabis plant), synthetic cannabinoids, cannabinoid mimetics, as well as salts, precursors, and metabolites thereof In some aspects, the compositions (e.g., complexes, nanoparticles, syrups) described herein are substantially free of or free of any cannabinoid.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as 2_{H}, 3_{H}, 13_{C}, 14_{C}, 15_{N}, 18_{O}, 17_{O}, 35_{S}, 18_{F} and 36_{Cl}, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as 3_{H} and 14_{C} are incorporated, are useful in drug and/or substrate tissue distribution assays. Tritiated, i.e., 3_{H}, and carbon-14, i.e., 14_{C}, isotopes are particularly preferred for their ease of preparation and detectability. Further, substitution with heavier isotopes such as deuterium, i.e., 2_{H}, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

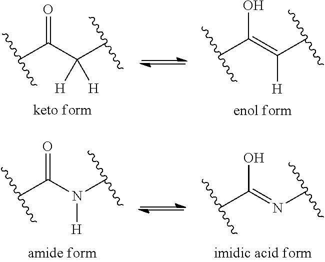 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

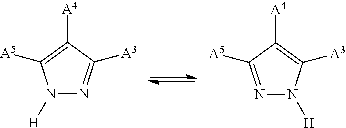

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process in vitro, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The subject can also be non-mammallian, e.g., a parakeet or a zebrafish. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a clinical, medical, or physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition) , and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelators and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cluster of differentiation 47 (CD47) mAB, toll-like receptor (TLR) agonists and other immune modifiers, cell therapeutics; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, nutraceuticals, nootropics, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes in some aspects, medicaments; vitamins; nutraceuticals (and in some instances the term “therapeutic agent” does not include a nutraceutical); mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. The term “therapeutic agent” also encompasses agents such as plasma, testestorone, and the like.

The term “nutraceutical” means a pharmaceutical alternative that has physiological benefits or for which physiological benefits have been reported. Nutraceuticals include without limitation a therapeutic agent (in some aspects), drug, dietary supplement, food ingredient, nootropic, or food. In some instances, a “nutraceutical” can be a therapeutic agent, and in other instances, “nutraceutical” is not a therapeutic agent.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “stable,” as used herein, refers to compounds or compositions that are not substantially altered when subjected to conditions to allow for their production, detection, and, in certain aspects, their recovery, purification, and use for one or more of the purposes disclosed herein. The phrase “substantially altered,” as used herein in this context, includes instances in which the compound or therapeutic agent or nutraceutical undergoes a change in molecular composition, isomerization, or a loss of therapeutic or physiological effect.

The therapeutic agents or nutraceuticals in some aspects may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

Therapeutic agents or nutraceuticals described herein can comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as 2_{H}, 3_{H}, 13_{C}, 14_{C}, 15_{N}, 18_{O}, 17_{O}, 35_{S}, 18_{F} and 36_{Cl}, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as 3_{H} and 14_{C} are incorporated, are useful in drug and/or substrate tissue distribution assays. Tritiated, i.e., 3_{H}, and carbon-14, i.e., 14_{C}, isotopes are particularly preferred for their ease of preparation and detectability. Further, substitution with heavier isotopes such as deuterium, i.e., 2_{H}, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The therapeutic agents or nutraceuticals in some aspects can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Materials, compounds, compositions, and components disclosed herein can also be prepared via bioprocesses.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Methods for Making the Therapeutic Agent or Nutraceutical Compositions

In one aspect, this disclosure relates to a method for making a therapeutic agent or nutraceutical composition, which generally comprises recovering the composition from a dispersion comprising: (a) a sugar, sugar alcohol, or sugar substitute; (b) water; and (c) an effective amount of the therapeutic agent or nutraceutical.

### 1. Preparing the Dispersion

In various aspects, the dispersion can be prepared by mixing the desired amount of sugar, sugar alcohol, or sugar substitute, water, and therapeutic agent or nutraceutical. In one aspect, the sugar, sugar alcohol, or sugar substitute can first be mixed with water, followed by addition of the desired amount of therapeutic agent or nutraceutical. In one aspect, the water can be distilled, filtered, or otherwise purified to remove impurities typically present in tap water.

In one aspect, the water used for preparing the dispersion can distilled, filtered, or otherwise purified and have a suitable pH. In a further aspect, the purified water used to prepare the dispersion can have a pH of from about 5 to about 9. In a further aspect, the purified water used to prepare the dispersion can have a pH of from about 5.5 to about 9. In a further aspect, the purified water used to prepare the dispersion can have a pH of from about 6.7 to about 9. In a further aspect, the purified water used to prepare the dispersion can have a pH of from about 6.8 to about 9. In a further aspect, the purified water used to prepare the dispersion can have a pH of from about 6 to about 9. In a still further aspect, the purified water used to prepare the dispersion can have a pH of from about 7 to about 8. In yet a further aspect, the purified water used to prepare the dispersion can have a pH within or near physiological limits, i.e., about 7 to about 8, or about 7.2 to about 7.5. In another aspect, the purified water used to prepare the dispersion can have a pH within any physiological limit.

In some aspects, e.g., when a therapeutic agent or nutraceutical syrup is prepared, the water used for preparing the dispersion can be fruit or plant water, including water derived from a vegetable produced from a plant. Non-limiting examples of suitable fruit waters include coconut water, pineapple water, cherry water, mango water, apple water, pomegranate water, and the like. Non-limiting examples of suitable plant or vegetable waters include cactus water, aloe vera water, beet water, carrot water, and the like. In some aspects, the fruit or plant water can comprise from about 70% to about 98% water by weight. In a further aspect, the fruit or plant water comprising from about 70% to about 98% water by weight can be further diluted with additional water.

According to one aspect, for a therapeutic agent or nutraceutical syrup, the water used for preparing the dispersion can be coconut water. In some aspects, the coconut water can comprise from about 85% to about 95% water, together with other known ingredients of coconut water. In some aspects, the coconut water can comprise from about 85% to about 95% water, together with one or more sugars including fructose, glucose or sucrose, in addition to various minerals including potassium, sodium magnesium, calcium, and iron, plus additional proteins and phenols. In one aspect, the coconut water comprises from about 10% to about 20% by weight potassium, about 1% to about 5% by weight calcium, and about 1% to about 4% by weight magnesium.

In one aspect, the dispersion can be prepared in a vessel comprising a surface that will not adhere to the therapeutic agent or nutraceutical, e.g., stainless steel. The inventors have discovered that the use of vessels comprising Teflon and silicone results in the therapeutic agent or nutraceutical adhering to the Teflon or silicone. Similarly, in various aspects, any utensils used for stirring, mixing, or agitating the dispersion can be stainless steel or glass, as the inventors have discovered that the use of wooden utensils can result in the therapeutic agent or nutraceutical sticking to the wood.

In various aspects, the mass ratio of the sugar, sugar alcohol, or sugar substitute to the water can vary generally depending on the amount of therapeutic agent or nutraceutical desired. In one aspect, for example, the mass ratio of the sugar, sugar alcohol, or sugar substitute to the water is from about 1:8 to about 1:1 prior to the recovering step, i.e., before the volume of the dispersion is reduced during any heating step. In another aspect, for example, the mass ratio of the sugar, sugar alcohol, or sugar substitute to the water is from about 1:8 to about 1:1.2 prior to the recovering step, i.e., before the volume of the dispersion is reduced during any heating step. In a further aspect, the mass ratio of the sugar, sugar alcohol, or sugar substitute to the water is from about 1:8 to about 1:1.3 prior to the recovering step. In a still further aspect, the mass ratio of the sugar, sugar alcohol, or sugar substitute to the water is from about 1:8 to about 1:1.5 prior to the recovering step. In yet a further aspect, the mass ratio of the sugar, sugar alcohol, or sugar substitute to the water can be about 1:8, 1:7, 1:6, 1:5, 1:4, 1:3, 1:2, 1:1.5, 1:1.3, 1:1.2, or 1:1 prior to recovering the therapeutic agent or nutraceutical composition from the dispersion. In some aspects, the mass ratio of the sugar, sugar alcohol, or sugar substitute to the water is from about 1:6 to about 1:4 prior to the recovering step. In one aspect, the mass ratio of the sugar, sugar alcohol, or sugar substitute to the water is from about 1:4 prior to the recovering step.

In one aspect, for example, it is contemplated that when the amount of therapeutic agent or nutraceutical added to the dispersion ranges from about 2-6 grams, the mass ratio of the sugar, sugar alcohol, or sugar substitute to the water can be about 3:4 (or about 1:1.33) or less. In a further aspect, when the amount of therapeutic agent or nutraceutical added to the dispersion is 6 grams or above (e.g., 6-12 grams), the mass ratio of the sugar, sugar alcohol, or sugar substitute to the water can be at least about 1:4. In other words, according to some aspects, more water can be added to the dispersion as the amount of therapeutic agent or nutraceutical added to the dispersion increases. Amounts of the therapeutic agent or nutraceutical and other components of the dispersion can be scaled up as desired.

The mass ratio of the therapeutic agent or nutraceutical to the sugar, sugar alcohol, or sugar substitute in the dispersion can vary. In one aspect, the mass ratio of the therapeutic agent or nutraceutical to the sugar, sugar alcohol, or sugar substitute is from about 1:300 to about 1:5. In another aspect, the mass ratio of the therapeutic agent or nutraceutical to the sugar, sugar alcohol, or sugar substitute is from about 1:50 to about 1:5. In a further aspect, the mass ratio of the therapeutic agent or nutraceutical to the sugar, sugar alcohol, or sugar substitute is from about 1:50 to about 1:10. In another aspect, the mass ratio of the therapeutic agent or nutraceutical to the sugar, sugar alcohol, or sugar substitute is from about 1:40 to about 1:10. In a further aspect, the mass ratio of the therapeutic agent or nutraceutical to the sugar, sugar alcohol, or sugar substitute is from about 1:30 to about 1:10. In a still further aspect, the mass ratio of the therapeutic agent or nutraceutical to the sugar, sugar alcohol, or sugar substitute is from about 1:30 to about 1:15. For example, the mass ratio of the therapeutic agent or nutraceutical to the sugar, sugar alcohol, or sugar substitute can be about 1:50, 1:45, 1:40, 1:35, 1:30, 1:25, 1:20, 1:18, 1:16, 1:15, 1:13, 1:12, 1:11, 1:10, 1:9, 1:8, 1:7, or 1:5. In one aspect, the mass ratio of the therapeutic agent or nutraceutical to the sugar, sugar alcohol, or sugar substitute is about 1:20.

### 2. Recovering the Therapeutic Agent or Nutraceutical Composition

In one aspect, the dispersion comprising the sugar, sugar alcohol, or sugar substitute, water, and therapeutic agent or nutraceutical can be heated at a temperature equivalent to from about 90° C. to about 122° C. at a pressure of 1 atm for a sufficient time to reduce the volume of the dispersion. As the volume of the dispersion decreases, the therapeutic agent or nutraceutical composition can solidify from the dispersion, e.g., by precipitation or coprecipitation of the therapeutic agent or nutraceutical and the sugar, sugar alcohol, or sugar substitute, thereby forming the therapeutic agent or nutraceutical complex or nanoparticle composition. In a further aspect, the dispersion comprising the sugar, sugar alcohol, or sugar substitute, water, and therapeutic agent or nutraceutical can be heated at a temperature equivalent to from about 104° C. to about 116° C. at a pressure of 1 atm. In a still further aspect, the dispersion comprising the sugar, sugar alcohol, or sugar substitute, water, and therapeutic agent or nutraceutical can be heated at a temperature equivalent to from about 107° C. to about 110° C. at a pressure of 1 atm. In yet another aspect, the dispersion comprising the sugar, sugar alcohol, or sugar substitute, water, and therapeutic agent or nutraceutical can be heated at a temperature equivalent to a temperature at a pressure of 1 atm sufficient to induce the dispersion to boil for a sufficient time to reduce the volume of the dispersion and thereby solidify the therapeutic agent or nutraceutical complex or nanoparticle composition. In other aspects, the process can result in a therapeutic agent or nutraceutical syrup and thus the dispersion may not solidify.

In some aspects, when the dispersion is heated to a temperature sufficient to induce the dispersion to boil for a sufficient amount of time at a stated pressure, equivalent boiling temperatures at different pressures can be calculated according to the Clausius-Clapeyron equation:

$$ln ( {P}_{2} ) - ln ( {P}_{1} ) = \frac{Δ {H}_{vap}}{R} [ \frac{1}{{T}_{1}} - \frac{1}{{T}_{2}} ]$$

where P₁ and T₁ are standard atmospheric pressure and the known boiling point of water, respectively, ΔHᵥₐₚ is the enthalpy of vaporization of water, and R is the gas constant (8.3145 J/mol*K). Using the Clausius-Clapeyron equation, for example, it can be determined that the boiling temperature of water at 2 atm of pressure (about 119° C.) can be equivalent to the boiling temperature of water at 1 atm of pressure (about 100° C.). Other known methods for determining temperatures equivalent to stated temperatures at stated atmospheric pressures can also be used.

The dispersion can be heated to the desired temperature or temperature range using methods known in the art. In one aspect, for example, the dispersion can be heated in a suitable vessel (e.g., a stainless steel vessel) by a suitable heat source, such as, for example, an induction cooktop. The heat source such as an induction cooktop can be maintained at a suitable temperature or temperature range such that the temperature of the dispersion stays at a temperature equivalent to from about 90° C. to about 122° C. at a pressure of 1 atm. Temperature of the dispersion can be monitored during the heating step using a thermometer, thermocouple, or other suitable device. The dispersion can also be heated under pressure at a suitable temperature equivalent to those described herein. In some aspects, the dispersion can be heated under reduced pressure at a suitable temperature equivalent to those described herein.

In various aspects, once the dispersion is heated for a sufficient time at the desired temperature, recovery of the therapeutic agent or nutraceutical composition from the dispersion can include various steps. In some aspects, for example, the therapeutic agent or nutraceutical composition can solidify or precipitate from the dispersion as the volume of the dispersion reduces to a certain level during heating. In one aspect, the dispersion can be heated at a temperature equivalent to from about 90° C. to about 122° C. at a pressure of 1 atm (e.g., equivalent to from about 104° C. to about 116° C. at 1 atm, or equivalent to from about 107° C. to about 110° C. at 1 atm) for a time sufficient to reduce the volume of the dispersion by about 10-95%, e.g., about 10%, 20%, 30%, 40%, 50%, 60%, 70%, 75%, 80%, 85%, 90%, or 95%, to thereby solidify or precipitate the therapeutic agent or nutraceutical composition from the dispersion.

In other aspects, once the dispersion is heated for a sufficient time at the desired temperature, recovery of the therapeutic agent or nutraceutical syrup from the dispersion can include various steps. In some aspects, for example, the therapeutic agent or nutraceutical syrup can form from the dispersion as the volume of the dispersion reduces to a certain level during heating. In one aspect, the dispersion can be heated at a temperature equivalent to from about 90° C. to about 122° C. at a pressure of 1 atm (e.g., equivalent to from about 104° C. to about 116° C. at 1 atm, or equivalent to from about 107° C. to about 110° C. at 1 atm) for a time sufficient to reduce the volume of the dispersion by about 10-95%, e.g., about 10%, 20%, 30%, 40%, 50%, 60%, 70%, 75%, 80%, 85%, 90%, or 95%, to thereby forming the therapeutic agent or nutraceutical syrup.

Optionally, as the volume of the dispersion comprising the sugar, sugar alcohol, or sugar substitute, water, and therapeutic agent or nutraceutical decreases during the heating step, various additional steps can be performed to aid in the recovery of the therapeutic agent or nutraceutical composition from the dispersion. In one aspect, for example, when the volume of the dispersion decreases to the desired level during the heating step, the dispersion can be agitated or stirred while maintaining the dispersion at the desired temperature or temperature range. In a further aspect, when the volume of the dispersion decreases to the desired level during the heating step, the dispersion can be agitated or stirred but not so vigorously as to create a vortex in the dispersion, while maintaining the dispersion at the desired temperature or temperature range. In one aspect, for example, the dispersion comprising the sugar, sugar alcohol, or sugar substitute, water, and therapeutic agent or nutraceutical can be agitated or stirred when the volume of the dispersion has decreased to the desired level, e.g., decreased by about 10-95%, e.g., about 10%, 20%, 30%, 40%, 50%, 60%, 70%, 75%, 80%, 85%, 90%, or 95%, to thereby solidify or precipitate the therapeutic agent or nutraceutical composition from the dispersion.

In other aspects, as the volume of the dispersion comprising the sugar, sugar alcohol, or sugar substitute, water, and therapeutic agent or nutraceutical decreases during the heating step, various additional steps can be performed to aid in the recovery of the therapeutic agent or nutraceutical syrup from the dispersion. In one aspect, for example, when the volume of the dispersion decreases to the desired level during the heating step, the dispersion can be agitated or stirred while maintaining the dispersion at the desired temperature or temperature range. In a further aspect, when the volume of the dispersion decreases to the desired level during the heating step, the dispersion can be agitated or stirred but not so vigorously as to create a vortex in the dispersion, while maintaining the dispersion at the desired temperature or temperature range. In one aspect, for example, the dispersion comprising the sugar, sugar alcohol, or sugar substitute, water, and therapeutic agent or nutraceutical can be agitated or stirred when the volume of the dispersion has decreased to the desired level, e.g., decreased by about 10-95%, e.g., about 10%, 20%, 30%, 40%, 50%, 60%, 70%, 75%, 80%, 85%, 90%, or 95%, to thereby form the therapeutic agent or nutraceutical syrup.

In some aspects, once formation of the therapeutic agent or nutraceutical syrup has been observed, the dispersion can remain at the desired temperature equivalent to from about 90° C. to about 122° C. at 1 atm (e.g., equivalent to from about 104° C. to about 116° C. at 1 atm, or equivalent to from about 107° C. to about 110° C. at 1 atm) for a time sufficient to evaporate most or all of the liquid remaining in the dispersion, leaving the therapeutic agent or nutraceutical syrup as a residue of the dispersion. In another aspect, once a syrup consistency has been achieved, the dispersion can be removed from the heat source with continuous agitation or stirring until the liquid remaining in the dispersion evaporates and the dispersion slowly cools. According to one aspect, for example, when the dispersion begins to attain the consistency of a slurry, the dispersion can be removed from the heat source and continuously agitated or stirred. Additional stirring or agitation can be performed until the slurry attains the consistency of a syrup. The resulting syrup can be dried as desired according to methods known in the art, e.g., drying under reduced pressure.

In one aspect, as the volume of the dispersion comprising the sugar, sugar alcohol, or sugar substitute, water, and therapeutic agent or nutraceutical decreases during the heating step, solidification of the therapeutic agent or nutraceutical composition from the dispersion can be aided by the use of an additive such as an antisolvent, e.g., a solvent that will not readily dissolve the therapeutic agent or nutraceutical composition.

In some aspects, once solidification of the therapeutic agent or nutraceutical composition from the dispersion has been observed, the dispersion can remain at the desired temperature equivalent to from about 90° C. to about 122° C. at 1 atm (e.g., equivalent to from about 104° C. to about 116° C. at 1 atm, or equivalent to from about 107° C. to about 110° C. at 1 atm) for a time sufficient to evaporate most or all of the liquid remaining in the dispersion. In another aspect, once solidification has been observed, the dispersion can be removed from the heat source with continuous agitation or stirring until the liquid remaining in the dispersion evaporates and the dispersion slowly cools. According to one aspect, for example, when the dispersion begins to attain the consistency of a slurry, the dispersion can be removed from the heat source and continuously agitated or stirred until the slurry resembles wet sand. Additional stirring or agitation can be performed until the slurry comprising the therapeutic agent or nutraceutical composition dries to a solid, granular form. Alternatively, according to other aspects, once solidification of the therapeutic agent or nutraceutical composition occurs, e.g., when the dispersion begins to attain the consistency of a slurry, the dispersion can be filtered through a suitable filter to provide a filtride comprising the therapeutic agent or nutraceutical composition. Similarly, in some aspects, once solidification of the therapeutic agent or nutraceutical composition has occurred, the dispersion can be dried according to methods known in the art, e.g., drying under reduced pressure.

Optionally, depending on the desired application, the dried therapeutic agent or nutraceutical composition obtained from the dispersion can be ground into a powder. A powder of the therapeutic agent or nutraceutical composition can be formed using methods known in the art, such as for example through the use of a food processor. In some aspects, once the therapeutic agent or nutraceutical composition has been recovered from the dispersion, the solid therapeutic agent or nutraceutical composition can be stored in a cool, dark, and dry environment until further use. In a further aspect, once the therapeutic agent or nutraceutical composition has been recovered from the dispersion, the solid therapeutic agent or nutraceutical composition can be refrigerated until further use, for example, if the therapeutic agent or nutraceutical composition comprises insulin. In one aspect, for example, the therapeutic agent or nutraceutical composition can be stored at a temperature equivalent to about 25° C. or less at a pressure of 1 atm (i.e., room temperature or below).

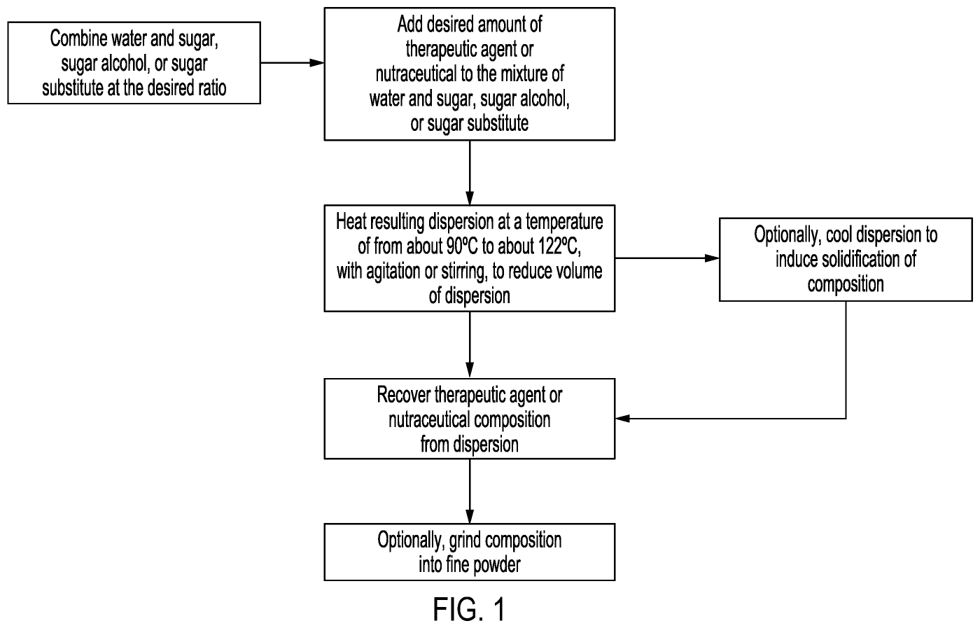

Referring now to FIG. 1, in one aspect, the method for making the therapeutic agent or nutraceutical complex or nanoparticle composition can comprise combining water and the sugar, sugar alcohol, or sugar substitute at the desired ratio, followed by adding the desired amount of therapeutic agent or nutraceutical to the mixture of water and the sugar, sugar alcohol, or sugar substitute. The resulting dispersion can be heated at a temperature equivalent to from about 90° C. to about 122° C. at a pressure of 1 atm for a sufficient time to reduce the volume of the dispersion. As the volume of the dispersion decreases, the therapeutic agent or nutraceutical complex or nanoparticle composition can solidify from the dispersion, depending on the dispersion composition. Additionally, in some aspects, the dispersion can be cooled to induce solidification of the therapeutic agent or nutraceutical complex or nanoparticle composition. Once solidification of the therapeutic agent or nutraceutical complex or nanoparticle composition has occurred, the complex or nanoparticle composition can be recovered from the dispersion and optionally ground into a fine powder suitable for use in a therapeutic product or dosage form such as those described below.

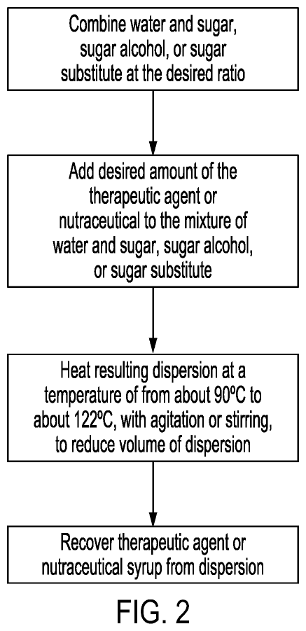

Referring now to FIG. 2, in one aspect, the method for making the therapeutic agent or nutraceutical syrup can comprise combining water (e.g., fruit or plant water such as coconut water) and the sugar, sugar alcohol, or sugar substitute at the desired ratio, followed by adding the desired amount of therapeutic agent or nutraceutical to the mixture of water and the sugar, sugar alcohol, or sugar substitute. The resulting dispersion can be heated at a temperature equivalent to from about 90° C. to about 122° C. at a pressure of 1 atm for a sufficient time to reduce the volume of the dispersion. As the volume of the dispersion decreases, the therapeutic agent or nutraceutical syrup can form from the dispersion. Once a syrup consistency has been achieved, the therapeutic agent or nutraceutical syrup can be recovered from the dispersion.

In one aspect, the therapeutic agent or nutraceutical syrup can have a range of viscosities. In some aspects, the therapeutic agent or nutraceutical syrup can be a solution or mixture comprising the therapeutic agent or nutraceutical together with a sugar, sugar alcohol, or sugar substitute, having a viscosity ranging from about 15 cp to about 25,000 cp. In a further aspect, the therapeutic agent or nutraceutical syrup can be a solution or mixture comprising the therapeutic agent or nutraceutical together with a sugar, sugar alcohol, or sugar substitute, having a viscosity ranging from about 100 cp to about 25,000 cp. In a still further aspect, the syrup can have a viscosity ranging from about 1,000 cp to about 25,000 cp. In yet a further aspect, the syrup can have a viscosity ranging from about 1,000 cp to about 10,000 cp. In another aspect, the syrup can have a viscosity ranging from about 1,000 cp to about 8,000 cp. In a further aspect, the syrup can have a viscosity ranging from about 1,000 cp to about 5,000 cp. In yet a further aspect, the syrup can have a viscosity ranging from about 1,000 cp to about 3,500 cp.

### 3. Composition of the Dispersion

In one aspect, the therapeutic agent or nutraceutical complexes can be water- and/or alcohol-soluble. Formation of the water- and/or alcohol-soluble therapeutic agent or nutraceutical complex can be obtained from a dispersion that is substantially free of any solvent other than water, e.g., a dispersion comprising less than about 10% by weight of any non-water-based solvent such as an organic solvent (e.g., hexane, alcohols, and the like). For example, according to one aspect, the dispersion can be substantially free of alcohol, e.g., comprise less than about 10% by weight of alcohol, relative to the total weight of the dispersion. Thus, in some aspects, the therapeutic agent or nutraceutical complex can be recovered from the dispersion without the use of an alcohol such as isopropyl alcohol or ethanol.

In a further aspect, the dispersion comprising the sugar, sugar alcohol, or sugar substitute, water, and therapeutic agent or nutraceutical can be substantially free of ethanol. In one aspect, the dispersion can comprise less than about 10%, less than about 5%, less than about 1%, less than about 0.5%, less than about 0.1%, less than about 0.05%, or less than about 0.01% of ethanol.

In other aspects, the dispersion comprising the sugar, sugar alcohol, or sugar substitute, water, and therapeutic agent or nutraceutical can be substantially free of isopropyl alcohol. In one aspect, for example, the dispersion can comprise less than about 10%, less than about 5%, less than about 1%, less than about 0.5%, less than about 0.1%, less than about 0.05%, or less than about 0.01% of isopropyl alcohol.

In a further aspect, the dispersion comprising the sugar, sugar alcohol, or sugar substitute, water, and therapeutic agent or nutraceutical can be substantially free of ethanol and isopropyl alcohol, e.g., comprise less than about 10%, less than about 5%, less than about 1%, less than about 0.5%, less than about 0.1%, less than about 0.05%, or less than about 0.01% of ethanol and isopropyl alcohol.

In another aspect, the dispersion can be free of ethanol. In a further aspect, the dispersion can be free of isopropyl alcohol. In a still further aspect, the dispersion can be free of ethanol and isopropyl alcohol. Similarly, in some aspects, the dispersion can be free of any organic solvent, including for example hexane or alcohol-based solvents.

In one aspect, the therapeutic agent or nutraceutical composition can be recovered from the dispersion comprising the sugar, sugar alcohol, or sugar substitute, water, and therapeutic agent or nutraceutical without the use of a carrier oil to improve the solubility of the therapeutic agent or nutraceutical, e.g., without creating an emulsion or other multi-phase system in the dispersion. Thus, in some aspects, the dispersion comprising the sugar, sugar alcohol, or sugar substitute, water, and therapeutic agent or nutraceutical is free of any carrier oil, including but not limited to medium chain triglyceride (MCT) oil, long chain triglyceride (LCT) oil, vegetable oil, canola oil, olive oil, sunflower oil, coconut oil (including fractionated coconut oil), hemp oil, palm oils, and/or other oils suitable for human or animal consumption. Similarly, the dispersion comprising the sugar, sugar alcohol, or sugar substitute, water, and therapeutic agent or nutraceutical can be free of other water-soluble agents used for creating an emulsion in the dispersion or improving the solubility of the therapeutic agent or nutraceutical, including without limitation a starch such as a modified food starch, gum arabic, quillaj a extract, or cyclodextrin. In some aspects, the dispersion can be similarly free of a sugar alcohol or maltodextrin, as further described below.

In a further aspect, the dispersion from which the therapeutic agent or nutraceutical composition can be recovered consists essentially of the sugar, sugar alcohol, or sugar substitute, the water, and the therapeutic agent or nutraceutical. In one aspect, as discussed below, the dispersion is free of a sugar alcohol, and thus the dispersion can consist essentially of the sugar or sugar substitute, the water, and the therapeutic agent or nutraceutical. Similarly, in some aspects, the dispersion is free of a sugar alcohol and sugar substitute and consists essentially of the sugar, the water, and the therapeutic agent or nutraceutical.

In a still further aspect, the dispersion from which the therapeutic agent or nutraceutical composition can be recovered consists of the sugar, sugar alcohol, or sugar substitute, the water, and the therapeutic agent or nutraceutical. In one aspect, as discussed below, the dispersion is free of a sugar alcohol, and thus the dispersion can consist of the sugar or sugar substitute, the water, and the therapeutic agent or nutraceutical. Similarly, in some aspects, the dispersion is free of a sugar alcohol and sugar substitute and consists of the sugar, the water, and the therapeutic agent or nutraceutical.

### a. Sugars, Sugar Alcohols, and Sugar Substitutes

A variety of sugars, sugar alcohols, and sugar substitutes are contemplated for use in the dispersion. In one aspect, the sugar, when present in the dispersion, can comprise allulose, glucose, dextrose, fructose, galactose, sucrose, lactose, maltose, trehalose, maltodextrin, or a combination thereof In a further aspect, the sugar, when present in the dispersion, can comprise sucrose, fructose, glucose, or a combination thereof. In a still further aspect, the sugar, when present in the dispersion, can comprise sucrose, fructose, and glucose. In one aspect, for example, the sugar can be a naturally-occurring sugar, such as cane sugar, which comprises sucrose, fructose, and glucose.

In one aspect, sugar alcohols suitable for use with the dispersion can include without limitation ethylene glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, polyglycitol, or a combination thereof In a further aspect, the sugar alcohol, when present in the dispersion, can comprise erythritol, xylitol, or a combination thereof

In another aspect, the dispersion is substantially free of a sugar alcohol. According to one aspect, for example, the dispersion can comprise less than about 10%, less than about 5%, less than about 1%, less than about 0.5%, less than about 0.1%, less than about 0.05%, or less than about 0.01% of any sugar alcohol. In one aspect, the dispersion can be substantially free of glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, and polyglycitol. In a further aspect, the dispersion can be substantially free of isomalt, mannitol, sorbitol, xylitol, lactitol, maltitol, and erythritol.

In one aspect, the dispersion is free of a sugar alcohol. Thus, for example, according to one aspect, the dispersion is free of glycol, glycerol, erythritol, threitol, arabitol, xylitol, ribitol, mannitol, sorbitol, galactilol, fucitol, iditol, inositol, volemitol, isomalt, maltitol, lactitol, maltotriitol, maltotetraitol, and polyglycitol. In a further aspect, the dispersion is free of isomalt, mannitol, sorbitol, xylitol, lactitol, maltitol, and erythritol.

A variety of sugar substitutes are also contemplated for use with the dispersion. Suitable plant-derived sugar substitutes include without limitation brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, xylitol (also known as a sugar alcohol), or a combination thereof

Suitable artificial sugar substitutes contemplated for use with the dispersion include without limitation acesulfame potassium, advantame, alitame, aspartame, salts of aspartame-acesulfame, sodium cylclamate, dulcin, glucin, neohesperidin dihidryochalcone, neotame, P-4,000, saccharin, sucralose, or a combination thereof In a further aspect, the dispersion can comprise an artificial sweetener comprising sucralose.

In another aspect, the dispersion is substantially free of any sugar substitute. According to one aspect, for example, the dispersion can comprise less than about 10%, less than about 5%, less than about 1%, less than about 0.5%, less than about 0.1%, less than about 0.05%, or less than about 0.01% of any sugar substitute. In one aspect, the dispersion can be substantially free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be substantially free of acesulfame potassium, advantame, alitame, aspartame, salts of aspartame-acesulfame, sodium cylclamate, dulcin, glucin, neohesperidin dihidryochalcone, neotame, P-4,000, saccharin, and sucrolose.

In another aspect, the dispersion is free of any sugar substitute. In one aspect, for example, the dispersion can be free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaultulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be free of acesulfame potassium, advantame, alitame, aspartame, salts of aspartame-acesulfame, sodium cylclamate, dulcin, glucin, neohesperidin dihidryochalcone, neotame, P-4,000, saccharin, and sucrolose.

### b. Therapeutic Agents and Nutraceuticals

In various aspects, the disclosed dispersions and resulting compositions include one or more therapeutic agents or nutraceuticals. The therapeutic agent or nutraceutical can be naturally-occurring, e.g., derived from a cell or plant, or can be produced synthetically. In a further aspect, the therapeutic agent or nutraceutical can be produced by genetically-modified cells or microbial factories.

In some aspects, when the therapeutic agent is a solid at a temperature equivalent to 25° C. at a pressure of 1 atm, the therapeutic agent has a melting point of 70° C. to 390° C. In a further aspect, when the therapeutic agent is a solid at a temperature equivalent to 25° C. at a pressure of 1 atm, the therapeutic agent has a melting point of 100° C. to 390° C. In additional aspects, when the therapeutic agent is a liquid at a temperature equivalent to 25° C. at a pressure of 1 atm, the therapeutic agent has a boiling point of at least 130° C., e.g., 130° C. to 200° C.

In one aspect, the therapeutic agent or nutraceutical can be stable, i.e., will not degrade, at the temperatures used in the disclosed method. Thus, in some aspects, the therapeutic agent or nutraceutical can be stable for at least about 30 minutes when exposed to a temperature of from about 90° C. to about 122° C. at a pressure of 1 atm. In a further aspect, the therapeutic agent or nutraceutical can be stable for at least about 30 minutes when exposed to a temperature of from about 104° C. to about 116° C. at a pressure of 1 atm. In a still further aspect, the therapeutic agent or nutraceutical can be stable for at least about 30 minutes when exposed to a temperature of from about 107° C. to about 110° C. at a pressure of 1 atm.

The therapeutic agent or nutraceutical can have a variety of molecular weights. It is contemplated that both small molecules and larger molecules, e.g., biologics, peptides, amino acid chains, and the like, can be incorporated into the dispersion. Thus, according to one aspect, the therapeutic agent or nutraceutical has a molecular weight of about 6,000 daltons (Da) or less. In a further aspect, the therapeutic agent or nutraceutical has a molecular weight of about 1,000 daltons (Da) or less. In a still further aspect, the therapeutic agent or nutraceutical has a molecular weight of from about 150 g/mol to about 1,000 g/mol. In a still further aspect, the therapeutic agent or nutraceutical has a molecular weight of from about 150 g/mol to about 500 g/mol. In a further aspect, the therapeutic agent, when present, has a molecular weight of 120 to 1,000 g/mol. The units “g/mol” are equivalent to daltons (Da). In a further aspect, the therapeutic agent comprises at least one aromatic group, including for example, an aryl or heteroaryl group as defined above.

According to one aspect, the therapeutic agent or nutraceutical has an octanol-water partition coefficient (logP) of from about −5 to about 15. In a further aspect, the therapeutic agent or nutraceutical has an octanol-water partition coefficient (logP) of from about −2 to about 10. In another aspect, the therapeutic agent or nutraceutical has an octanol-water partition coefficient (logP) of from about −5 to about 8. In one aspect, the therapeutic agent has an octanol-water partition coefficient of −4.1 to 7.2. In a further aspect, the therapeutic agent or nutraceutical has a pKa of from about 1 to about 13, e.g., about 2, 3, 4, 5, 6, 7, 8, 9, 10, 11, 12, or 13. According to a further aspect, the therapeutic agent or nutraceutical has a water solubility of at least about 0.001 pg/mL.

The effective amount of the therapeutic agent or nutraceutical present in the dispersion can vary depending on the therapeutic agent or nutraceutical and its use. According to one aspect, the effective amount of the therapeutic agent or nutraceutical is a therapeutically effective amount. According to a further aspect, the effective amount of the therapeutic agent or nutraceutical is a prophylactically effective amount.

A wide variety of therapeutic agent or nutraceuticals can be incorporated into the disclosed dispersions to form a therapeutic agent or nutraceutical composition. According to one aspect, the therapeutic agent or nutraceutical is a medicament, vitamin, mineral supplement, or a substance used for the treatment, prevention, diagnosis, cure, or mitigation of a disease or illness. In one aspect, the therapeutic agent or nutraceutical is a compound or composition that can be administered orally.

In a further aspect, the therapeutic agent is a biologic, an adjuvant, anti-infective agent, anti-cancer agent, anti-neoplastic agent, poly ADP ribose polymerase (PARP) inhibitor, DNA damage response modifier, epigenetic agent, histone deacetylast (HDAc) inhibitor, iron chelator, ribonucleotide reductase inhibitor, proteasome inhibitor, Nedd8-activating enzyme (NAE) inhibitor, mammalian target of rapamycin (mTOR) inhibitor, cytotoxic agent, immune checkpoint blockade agent, toll-like receptor (TLR) agonist, immune modifier, cell therapeutic, anti-ALS agent, analgesic, anorexic, anti-inflammatory agent, anti-epileptic agent, anesthetic, hypnotic, sedative, antipsychotic agent, neuroleptic agent, antidepressant, anxiolytics, antagonist, neuron blocking agent, anticholinergic agent, cholinomimetic agent, antimuscarinic agent, muscarinic agent, antiadrenergic, antiarrhythmic, antihypertensive agent, hormone, nutrient, nutraceutical, antiarthrtic, antiasthmatic, anticonvulsant, antihistamine, antinauseant, antineoplastic, antipruritic, antipyretic, antispasmodic, cardiovascular preparation, diuretic, vasodilator, central nervous system stimulant, cough or cold agent, decongestant, diagnostic, bone growth stimulant, bone resorption inhibitor, immunosuppressive, muscle relaxant, psychostimulant, tranquilizer, pain or fever reducer, antiviral, antiretroviral, antibacterial, platelet reducer, anticoagulant, an agent for treating muscle spasms, or an agent for treating dry mouth.

According to one aspect, two or more therapeutic agents or nutraceuticals can be present in the dispersion. Alternatively, according to a further aspect, a therapeutic agent or nutraceutical composition can be prepared with a first therapeutic agent or nutraceutical, and one or more additional therapeutic agent or nutraceutical complexes or syrups can be prepared with additional therapeutic agent(s) or nutraceutical(s), and the resulting complexes or syrups can be mixed together to form a composite composition. Such a composite composition may be useful for example in preventing unwanted interaction between two or more therapeutic agents or nutraceuticals.

In one aspect, for example, a single composition or a composite of such complexes or syrups can comprise one or more anti-bacterial therapeutic agents, e.g., β-lactam antibiotic plus an aminoglycoside, which can be useful for subjects with bacteremia and neutropenia. Similarly, for example, infections with Pseudomonas aeruginosa or Acinetobacter baumannii isolates that are resistant to antibiotics except polymyxins, several antibiotic combinations can demonstrate increased activity compared with that of any single agent.

In a further aspect, the following combination therapies are contemplated for use with the therapeutic agent complexes or syrups: Pylera (bismuth sub/metronidazole/tetracycline), TRUVADA (emtricitabine/tenofovir), Sinemet (carbidopa/levodopa), BIKTARVY (bictegruvir/emtricitabine/tenofivir), Lisinopril-hydrochlorothiazide, Amlodipine/atorvastatin, Hydrocodome-Apap, Oxycodone-Apap, Buprenorphine-naloxone, Atenolol-chlorthalidome, Xigduo (Dapagliflozin/Metformin), various prenatal vitamins (e.g., Folic acid) comprising multiple vitamins/minerals/DHA, among other combination therapies known in the art.

According to a further aspect, the therapeutic agent or nutraceutical is not a cannabinoid. In other aspects, the therapeutic agent or nutraceutical is not an agent known for treating or preventing cough. According to one aspect, for example, the therapeutic agent or nutraceutical does not include dextromethorphan, camphor, eucalyptus oil, menthol, guaifenesin, or any combination thereof. In a further aspect, the therapeutic agent or nutraceutical composition does not comprise caffeine.

In a further aspect, the therapeutic agent can be a non-steroidal anti-inflammatory, e.g., aspirin, a carboxylic acid-containing therapeutic agent, an acidic or basic amino acid, Imatinib, doxycycline for a variety of subjects including mammals, e.g., pets or human subjects, levofloxacin, ivermectin, an anti-HIV drug, or vitamin such as Vitamin D.

In a still further aspect, the therapeutic agent can be any of the agents listed in Table 1A below together or in any logical or therapeutically useful combination. Any of the agents below can be present either in neutral or salt form, including without limitation any of the salt forms approved by the FDA. Thus, although certain salts and neutral counterparts are specifically described and listed, it is contemplated that when a neutral form is described, salts or the neutral form are described, and when a salt form is described, neutral counterparts of the salt are described.

TABLE 1A
EXEMPLARY THERAPEUTIC AGENTS

| | | Molecular |
|---|---|---|
| | | Weight |
| Compound | Use | (g/mol) |
| Acetaminophen | Pain/Fever Reduction | 151.163 |
| Acyclovir | Antiviral | 225.21 |
| Albuterol sulfate | Asthma | 576.7 |
| Amiodarone | Heart Rhythm | 645.31 |
| hydrochloride | | |
| Amoxicillin | Anti-bacterial | 365.4 |
| Amphetamine | ADD | 421.5 |
| aspartate | | |
| Anagrelide | Platelet Reducer | 292.5 |
| hydrochloride | | |
| Anastrozole | Breast Cancer | 293.374 |
| Apixaban | Anticoagulant | 459.5 |
| Apripiprazole | Atypical antipsychotic | 448.4 |
| Aspirin | Pain | 180.16 |
| Atenolol | Beta blocker | 266.34 |
| Baclofen | muscle spasms | 213.67 |
| Benztropine | Used to treat a type of movement | 307.44 |
| | disorder due to antipsychotics | |
| | known as dystonia and parkinsonism. | |
| | Benefits are seen within | |
| | 2 hours and last for up to 10 hours | |
| Bethanechol | dry mouth | 196.68 |
| chloride | | |
| (bethanechol) | | |
| Bisoprolol | High blood pressure | 325.44 |
| fumarate | | |
| Bisacodyl | Laxative | 361.39 |
| Brompheniramine | antihistamine | 435.3 |
| maleate | | |
| Budesonide | Crohn's disease and ulcerative colitis | 430.53 |
| Buspirone | Anxiety | 385.51 |
| Buspirone | Anxiety | 422 |
| hydrochloride | | |
| Butalbital | Pain | 224.26 |
| Carbamazepine | seizures, nerve pain, and | 236.27 |
| | bipolar disorder | |
| Carvedilol | Heart failure, left bentricular | 713.92 |
| | dysfunction, and hypertension. | |
| | Belongs to class of drugs known | |
| | as alpha and beta blockers. | |
| Cedazuridine | myelodysplastic syndromes (MDS) | 268.21 |
| Cetirizine | Second-generation histamine | 388.89 |
| | H1 antagonist | |
| Chlordiazepoxide | anxiety, alcohol withdrawal | 336.2 |
| Hydrochloride | symptoms, and tremor | |
| Chlorozoxazone | pain and stiffness caused by | 169.57 |
| | muscle spasm | |
| Chloroquine | Immunosuppressive and anti-parasite | 319.88 |
| Citalopram | Antidepressant | 324.4 |
| Clemastine | hay fever and allergy symptoms | 460 |
| fumarate | | |
| Clidinium bromide | peptic ulcer | 432.3 |
| Clopidogrel | Antiplatelet | 321.82 |
| Colchicine | Anti-inflammatory | 399.44 |
| Colesevelam | cholesterol levels and treat type | 618.2 |
| hydrochloride | 2 diabetes. | |
| Cyclobenzaprine | pain and discomfort related | 311.8 |
| hydrochloride | to muscle spasms, sprains and | |
| | other muscle injuries | |
| Cysteamine | Cystine reducer | 227.24 |
| bitartrate | | |
| Decitabine | myelodysplastic syndromes (MDS) | 228.21 |
| Deferasirox | chronic iron overload | 373.4 |
| Dextromethorphan, | Cough relief | 370.3 |
| hydrobromide | | |
| Diazoxide | low blood sugar (hypoglycemia) | 230.67 |
| Diphenylhydantoin | Anticonvulsant | 252.27 |
| Donepezil | Cognition-enhancing | 379.49 |
| Doxepin | depression, anxiety, and sleep | 315.8 |
| Hydrochloride | disorders | |
| Doxycycline | Anti-bacterial | 444.4 |
| Doxycycline | Anti-bacterial | 512.9 |
| hyclate | | |
| Doxylamine | hay fever and other allergy symptoms | 270.37 |
| Doxylamine | hay fever and other allergy symptoms | 388.5 |
| Succinate | | |
| Dutasteride | Urinary retention | 528.5 |
| D-pencillamine | Immunosuppressive drug and Heavy | 149.21 |
| | metal toxicity medication | |
| Eletriptan | migraine headaches | 463.4 |
| hydrobromide | | |
| Erlotinib | cancer, including pancreatic and | 429.9 |
| hydrochloride | non-small cell lung cancer | |
| Erythromycin | Antibiotics and Gut motility stimulator | 733.93 |
| Estazolam | insomnia | 294.7 |
| Ethinyl estradiol | estrogen medication | 296.4 |
| Everolimus | cancer of the kidney, pancreas, | 958.2 |
| | breast, and brain | |
| Famotidine | Pepetic ulcer disease, | 337.44 |
| | gastroesophageal reflux disease | |
| | (GERD), Zollinger-Ellison syndrome | |
| Fenofibrate | Cholesterol | 360.83 |
| Fingolimod | multiple sclerosis (MS) | 343.9 |
| Fluconazole | fungal infections | 306.27 |
| Fluphenazine | schizophrenia | 437.52 |
| Fluphenazine | schizophrenia | 510.4 |
| hydrochloride | | |
| Fluoxetine | Antidepressant, Separation Disorder | 345.79 |
| hydrochloride | in pets such as dogs | |
| Fostemsavir | antiretroviral medication (HIV) | 704.6 |
| tromethamine | | |
| Glimepiride | Anti-diabetic | 490.62 |
| Glipizide | Anti-diabetic | 445.54 |
| Griseofulvin | fungal infections | 352.76 |
| Guaifenesin | Expectorant (cough and cold) | 198.22 |
| Guanfacine | antihypertensive | 282.5 |
| Hydrochloride | | |
| Hydrochlorothiazide | high blood pressure and fluid | 297.74 |
| | retention (edema) | |
| Ibuprofen | Non-steroidal anti-inflammatory | 206.29 |
| Imatinib | Chronic lyelogenous, leukemia | 493.62 |
| | (CML), gastrointestinal | |
| | stromal tumors (GISTs), | |
| | and other malignancies | |
| Imatinib Mesylate | leukemia and other kinds of cancer | 589.7 |
| Insulin | Blood sugar control | 5734 |
| Irbesartan | Antihypertensive | 428.5 |
| Itraconazole | Fungal infections in immuno- | 705.64 |
| | compromised and non- | |
| | immunocompromised patients: | |
| | pulmonary and extrapulmonary | |
| | glastomycosis, histoplasmosis, | |
| | aspergillosis, and onychomycosis. | |
| Lamotrigine | Mood stabilizer, bipolar disorder, | 256.1 |
| | seizures | |
| Leucovorin | prevent the side effects of a | 473.44 |
| calcium | cancer medication | |
| Levothyroxine | Thyroid hormone deficiency, | 776.87 |
| | including myxedema coma. | |
| | Also used to treat and prevent | |
| | certain types of thyroid tumors. | |
| Liothyronine | Hypothyroidism | 650.97 |
| sodium | | |
| Lidocaine | Antiarrhythmic and Anesthetic | 234.34 |
| Levodopa | Parkinson's disease and sopamine- | 197.19 |
| | responsive dystonia | |
| Levofloxacin | Antibiotic to treat bacterial | 361.37 |
| | infections including acute | |
| | bacterial sinusitis, pneumonia, | |
| | urinary tract infections, chronic | |
| | prostatitis and some type of | |
| | gastroenteritis. Along with | |
| | other antibiotics, it may be | |
| | used to treat tuberculosis, meningitis | |
| | or pelvic inflammatory disease. | |
| Lisinopril dihydrate | ACEI inhibitor, hypertension, heart | 441.53 |
| | failure, myocardial infarction | |
| Loratadine | Allergies, including allergic rhinitis | 382.89 |
| | (hay fever) and hives | |
| Lovaststin | High cholesterol and triglyceride | 404.54 |
| Meloxicam | Nonsteroidal anti-inflammatory drug | 351.4 |
| Memantine | Cognition-enhancing medication | 179.3 |
| Mesalamine | Nonsteroidal anti-inflammatory drug | 153.13 |
| Metaxalone | Muscle relaxant | 221.25 |
| Methotrexate | cancer of the blood, bone, lung, | 454.44 |
| sodium | breast, head, and neck | |
| Methylphenidate | ADHD and narcolepsy | 233.31 |
| Metronidazole | Antibacterial, skin redness | 171.16 |
| Metyrosine | symptoms caused by adrenal gland | 195.22 |
| | tumors (pheochromocytoma) | |
| Mexitil (Mexiletine | irregular heartbeats (arrhythmias) | 215.72 |
| HCl) | | |
| Mifepristone | Progesterone blocker | 429.6 |
| Mycophenolate | prevent organ rejection after | 433.49 |
| Mofetil | transplant | |
| Naloxegol oxalate | constipation caused by opiate | 741.8 |
| | (narcotic) pain medications in | |
| | adults with chronic (ongoing) pain | |
| Naproxen | Nonsteroidal anti-inflammatory | 230.26 |
| Naloxone | Opiate overdose | 327.3 |
| Nifedipine | Antihypertensive drug and Calcium | 346.3 |
| | channel blocker | |
| Norgestimate | estrogen medication | 369.5 |
| Olanzapine | mental disorders, including | 312.4 |
| | schizophrenia and | |
| | bipolar disorder | |
| Olmesartan | high blood pressure | 558.59 |
| medoxomil | | |
| Ondanestron | nausea and vomiting | 293.4 |
| Oxycodone | Pain | 315.36 |
| hydrochloride | | |
| Penicillamine | Immunosuppressive drug and Heavy | 149.21 |
| | metal toxicity medication | |
| Phenytoin | Anticonvulsant | 252.27 |
| Piaglitazone | Anti-diabetic | 356.44 |
| Pioglitazone | Anti-diabetic medication | 392.9 |
| hydrochloride | | |
| Plavix | prevent stroke, heart attack, and | 419.9 |
| (Clopidogrel | other heart problems | |
| Bisulfate) | | |
| Prasugrel | Blood thinner | 409.9 |
| hydrochloride | | |
| Pravachol | high cholesterol and triglyceride | 446.5 |
| (Pravastatin | levels | |
| Sodium) | | |
| Prednisone | Immunosuppressant, | 358.43 |
| | inflammation, etc. | |
| Pregabalin | Nerve pain medication | 159.23 |
| Prucalopride | impaired motility associated | 486 |
| succinate | with chronic constipation | |
| Pyrazinamide | tuberculosis | 123.11 |
| Pyridoxine | dietary supplement | 205.64 |
| Hydrochloride | | |
| Ranitidine | Antihistamine and Antacid | 314.4 |
| Rosuvastatin | high cholesterol and triglyceride levels | 481.5 |
| Risperidone | Antipsychotic | 410.49 |
| Safinamide | idiopathic Parkinson's disease (PD) | 302.34 |
| Mesylate | | |
| Selegiline (BBB) | Parkinson's and major depressive | 187.29 |
| | disorder | |
| Sertraline | Antidepressant | 306.23 |
| Sildenafil | Vasodilator | 474.6 |
| Spironolactone | high blood pressure | 416.57 |
| Sulfamethoxazole | Antibiotic | 253.28 |
| Tadalafil | erectile dysfunction and enlarged | 389.4 |
| | prostate | |
| Tolcapone | symptoms of Parkinson's disease | 273.25 |
| Tolterodine tartrate | overactive bladder symptoms | 475.6 |
| Triheptanoin | long-chain fatty acid oxidation | 428.6 |
| | disorders | |
| Trimethoprim | Antibiotics and Folate synthesis | 290.32 |
| | inhibitor | |
| Ultramicrosize | antifungal | 352.8 |
| Vancomycin | Antibiotic | 1,449.30 |
| Vardenafil | Erectile dysfunction | 525.1 |
| Venlafaxine | Antidepressant and Nerve pain | 277.4 |
| | medication | |
| Voriconazole | fungal infections | 349.31 |
| Warfarin | Blood thinner | 308.33 |
| Zidovudine | HIV antiviral | 267.24 |

In one aspect, the nutraceutical can be any of the agents listed in Table 1B below either alone or in combination.

TABLE 1B
EXEMPLARY NUTRACEUTICALS

| Nutraceutical | Reported Benefits |
|---|---|
| Moringa | Anti-oxidant and anti-inflammatory |
| Vitamin D3 | Various |
| L-Theanine | Anti-anxiety |
| Methylsulfonylmethane | Anti-inflammatory |
| (MSM) | |
| Asian Ginseng | Performance |
| Guarana | Anti-oxidant and stimulant |
| Ginger Root | Gastroprotective, Anti-oxidant/anti- |
| | inflammatory, Neuroprotective, |
| | Cardioprotective |
| Ceylon Cinnamon | Various |
| Curcumin | Anti-inflammatory |
| Magnesium Oxide | Performance |
| Tumeric | Anti-inflammatory |
| Caralluma Fimbrita | Various |
| Beet Root | Various |
| Green Tea Extract | Various |
| Garcinia Cambogia | Various |
| Conjugated linoleic acid | Various |
| (CLA) | |
| CoQ10 | Anti-oxidant/anti-inflammatory |
| Maca | Various |
| Echinacea | Various |
| Nutraceutical | Reported Benefits |
| L-Tryptophan | Anti-anxiety |
| Coconut Oil | Various |
| Milk Thistle | Anti-oxidant |
| Lime | Various |
| Valerian Root | Anti-anxiety |
| St. John's Wort | Various |
| Kava Tincture | Various |
| Black seed oil | Various |
| Melatonin | Sleep aid |
| L-Carnitine | Various |
| L-Thyroxine | Various |
| Lemongrass | Various |
| Lemon | Various |
| Fennel | Various |
| Oregano | Various |
| Peppermint | Various |

According to one aspect, disclosed is a therapeutic agent or nutraceutical composition consisting essentially of a sugar, sugar alcohol, or sugar substitute; an effective amount of the therapeutic agent or nutraceutical; wherein the therapeutic agent or nutraceutical complex, when present, comprises less than 25% water by weight of the therapeutic agent or nutraceutical complex; and wherein the therapeutic agent or nutraceutical syrup, when present, comprises 30% to 60% water by weight of the therapeutic agent or nutraceutical syrup. In a still further aspect, the therapeutic agent or nutraceutical composition consists of the sugar, sugar alcohol, or sugar substitute; the effective amount of the therapeutic agent or nutraceutical water; wherein the therapeutic agent or nutraceutical complex, when present, comprises less than 25% water by weight of the therapeutic agent or nutraceutical complex; and wherein the therapeutic agent or nutraceutical syrup, when present, comprises 30% to 60% water by weight of the therapeutic agent or nutraceutical syrup. In a further aspect, the therapeutic agent or nutraceutical complex, when present, comprises 10% to 20% water by weight of the therapeutic agent or nutraceutical complex. In another aspect, the therapeutic agent or nutraceutical complex, when present, comprises 11% to 17% water by weight of the therapeutic agent or nutraceutical complex. Also disclosed are products comprising the therapeutic agent or nutraceutical composition.

### c. Acceptable Salts and Carriers

In some aspects, a naturally-occurring therapeutic agent or nutraceutical or sugar present in the dispersion or a product prepared therefrom can be present as an acceptable, non-naturally occurring salt. Thus, for example, a naturally-occuring therapeutic agent or nutraceutical or sugar present in the composition can be present as a non-naturally occurring acid or base salt of the naturally-occurring therapeutic agent or nutraceutical or sugar. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

Acceptable salts can be prepared by reaction of the therapeutic agent or nutraceutical or sugar with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates.

According to one aspect, if the therapeutic agent or nutraceutical or sugar has one or more acidic functional groups, the desired salt can be prepared by any suitable method known in the art, for example, treatment of the free acid with an inorganic or organic base, such as an amine (primary, secondary or tertiary), an alkali metal hydroxide or alkaline earth metal hydroxide, or the like. It is understood that the acceptable salts are non-toxic and suitable for ingestion. Additional information on suitable acceptable salts can be found in Remington's Pharmaceutical Sciences, 17th ed., Mack Publishing Company, Easton, Pa., 1985, which is incorporated herein by reference.

In some aspects, a product prepared from a disclosed dispersion can comprise a naturally-occurring therapeutic agent or nutraceutical and/or sugar present along with an acceptable, non-naturally occurring carrier. Various suitable non-naturally occurring carriers are described in Remington's Pharmaceutical Sciences, 17th ed., Mack Publishing Company, Easton, Pa., 1985, which is incorporated herein by reference. Non-limiting examples include non-naturally occurring polymeric carriers or binders in liquid or solid form, such as polyglycolic acids, synthetic polymers, non-naturally occurring conjugates of proteins, and the like.

### d. Exemplary Dispersions

Specific, non-limiting examples of dispersions useful for preparing the therapeutic agent or nutraceutical composition include those listed in Table 2. The compositions listed in Table 2 include dispersions comprising a sugar, sugar alcohol, or sugar substitute selected from a sugar comprising sucrose, fructose, and glucose (e.g., cane sugar or organic cane sugar); a sugar alcohol selected from erythritol or xylitol; maltodextrin, or sucralose. In one aspect, the exemplary dispersion compositions listed in Table 2 include a sugar comprising sucrose, fructose, and glucose such as cane sugar or organic cane sugar.

TABLE 2
EXEMPLARY DISPERSION COMPOSITIONS

| Mass Ratio of Therapeutic Agent | Mass Ratio of Sugar/ |
|---|---|
| or Nutraceutical to Sugar/Sugar | Sugar Alcohol/Sugar |
| Alcohol/Sugar Substitute | Substitute to Water |
| 1:300 to 1:5 | 1:8 to 1:1 |
| 1:300 to 1:5 | 1:8 to 1:1.2 |
| 1:300 to 1:5 | 1:8 to 1:1.3 |
| 1:300 to 1:5 | 1:8 to 1:1.5 |
| 1:300 to 1:5 | 1:6 to 1:4 |
| 1:300 to 1:5 | 1:4 |
| 1:50 to 1:5 | 1:8 to 1:1 |
| 1:50 to 1:5 | 1:8 to 1:1.2 |
| 1:50 to 1:10 | 1:8 to 1:1 |
| 1:50 to 1:10 | 1:8 to 1:1.2 |
| 1:40 to 1:10 | 1:8 to 1:1 |
| 1:40 to 1:10 | 1:8 to 1:1.2 |
| 1:30 to 1:10 | 1:8 to 1:1 |
| 1:30 to 1:10 | 1:8 to 1:1.2 |
| 1:30 to 1:10 | 1:8 to 1:1.2 |
| 1:30 to 1:15 | 1:8 to 1:1 |
| 1:30 to 1:15 | 1:8 to 1:1.2 |
| 1:20 | 1:8 to 1:1 |
| 1:20 | 1:8 to 1:1.2 |
| 1:50 to 1:10 | 1:8 to 1:1.3 |
| 1:50 to 1:10 | 1:8 to 1:1.5 |
| 1:50 to 1:10 | 1:6 to 1:4 |
| 1:50 to 1:10 | 1:4 |
| 1:30 to 1:10 | 1:8 to 1:1.3 |
| 1:30 to 1:10 | 1:8 to 1:1.5 |
| 1:30 to 1:10 | 1:6 to 1:4 |
| 1:30 to 1:10 | 1:4 |
| 1:30 to 1:15 | 1:8 to 1:1.3 |
| 1:30 to 1:15 | 1:8 to 1:1.5 |
| 1:30 to 1:15 | 1:6 to 1:4 |
| 1:30 to 1:15 | 1:4 |
| 1:20 | 1:8 to 1:1.3 |
| 1:20 | 1:8 to 1:1.5 |
| 1:20 | 1:6 to 1:4 |
| 1:20 | 1:4 |

### C. Products Comprising the Therapeutic Agent or Nutraceutical Compositions

Also described herein are products comprising the therapeutic agent or nutraceutical composition prepared by a disclosed method. One advantage of the disclosed method is that it allows for the infusion of a therapeutic agent or nutraceutical into a product such as a comestible product that masks the taste of the therapeutic agent or nutraceutical. In one aspect, for example, a water-based or alcohol-based comestible can comprise the therapeutic agent or nutraceutical composition prepared by a disclosed method, together with a water-based or alcohol-based liquid. In another aspect, the therapeutic agent or nutraceutical composition can be at least partially dissolved in the comestible liquid, e.g., water-based or alcohol-based liquid. In a further aspect, the comestible liquid such as a water-based or alcohol-based liquid can comprise the therapeutic agent or nutraceutical composition uniformly dispersed therein. In a further aspect, a solid or semi-solid comestible can comprise the therapeutic agent or nutraceutical composition prepared by a disclosed method, together with one or more foodstuff ingredients. In various aspects, the therapeutic agent or nutraceutical composition prepared by a disclosed method can be infused into a water-based or alcohol-based comestible by mixing the therapeutic agent or nutraceutical composition into the liquid, e.g., by stirring or agitating the mixture.

In further aspects, the therapeutic agent or nutraceutical composition prepared by a disclosed method can be incorporated into a product for therapeutic use, including products suitable for humans and animals. The therapeutic agent or nutraceutical complexes and syrups can be useful for treating a variety of conditions, disorders, and illnesses.

In various aspects, the therapeutic agent or nutraceutical composition prepared by a disclosed method can be formulated as a composition or formulation comprising a suitable carrier. Non-limiting examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and organic esters such as ethyl oleate. Proper fluidity can be maintained, for example, by the use of coating materials such as lecithin, by the maintenance of the required particle size in the case of dispersions and by the use of surfactants.

Suitable carriers for a product for therapeutic use can also comprise adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Suitable inert carriers can include sugars such as lactose.

In some aspects, the product for a therapeutic use can include an excipient. Suitable excipients include, without limitation, saccharides, for example, glucose, lactose, or sucrose, mannitol, or sorbitol, cellulose derivatives, and/or calcium phosphate, for example, tricalcium phosphate or acidic calcium phosphate.

In further aspects, the product for a therapeutic use can include a binder. Suitable binders include, without limitation, tare compounds such as starch paste, for example, corn, wheat, rice, and potato starch, gelatin, tragacanth, methylcellulose, hydroxypropyl methylcellulose, carboxymethylcellulose, and/or polyvinylpyrrolidone. In still further aspects, there can be a disintegrating agent, such as the aforementioned starches and carboxymethyl starch, crosslinked polyvinylpyrrolidone, agar, or alginic acid or a salt thereof, such as sodium alginate.

In some aspects, the product for a therapeutic use can include an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, or silica. In a further aspect, the additive can include flowability-control agents and lubricants, such as silicon dioxide, talc, stearic acid and salts thereof, such as magnesium stearate or calcium stearate, and/or propylene glycol.

The therapeutic product can be formulated for oral use, such as for example, a tablet, pill, or capsule, and the composition can include a coating layer that is resistant to gastric acid. Such a layer, in various aspects, can include a concentrated solution of saccharides that can comprise gum arabic, talc, polyvinylpyrrolidone, polyethylene glycol, and/or titanium dioxide, and suitable organic solvents or salts thereof

The effective amount of the therapeutic agent or nutraceutical in the therapeutic product can vary within wide limits. Such a dosage can be adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted in the event of any contraindications. Dosage can vary, and can be administered in one or more dose administrations daily, for one or several days.

### D. Examples

The following examples are put forth so as to provide those of ordinary skill in the art with a complete disclosure and description of how the methods and products claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. The Examples are provided herein to illustrate the invention, and should not be construed as limiting the invention in any way.

### 1. General Methods of Preparing Dispersions

To prepare the exemplary dispersions, an induction cooktop was used to maintain consistent heat, along with a stainless steel pot as a vessel for the dispersion, and stainless steel or glass utensils for stirring or agitation. The desired amount organic cane sugar and filtered or spring water was first mixed before the addition of the therapeutic agent or nutraceutical. A desired amount of therapeutic agent or nutraceutical was then added to the mixture, and the induction cooktop was set at a temperature range of from 100° C. to 138° C. It was observed that setting the temperature of the induction cooktop to a temperature above 138° C. can affect the quality of the composition recovered from the dispersion. The dispersion was then brought to a boil. After about ¾ of the volume of the original dispersion boiled away, the dispersion was agitated with stirring while keeping the dispersion heated on the induction cooktop. The dispersion was stirred and agitated but not so vigorously as to create a vortex in the dispersion.

Optionally, depending on the ratio of organic cane sugar to the water, an optional seeding step was performed. Incrementally, a pinch (about 0.36 grams) of organic cane sugar was added to the dispersion with stirring until visible solidification (e.g., precipitation) of the composition was observed. Or in some cases, the composition did not solidify and instead remained a syrup. The sides of the stainless steel vessel were also scraped to dislodge any solidified composition and in some cases to aid in further solidification of the composition.

The stainless steel vessel comprising the composition and remaining liquid was then removed from the induction cooktop when most of the liquid had evaporated from the dispersion and the dispersion resembled wet sand. The dispersion was continuously stirred until the remaining liquid evaporated, which yielded either granulated solid nanoparticles resembling dry sand, or in some instances, a syrup.

The composition recovered from the dispersion was then optionally ground into a fine powder using a food processor. The ground powder was stored in a dry, dark and cool location (less than about 75° F.) until further use.

### 2. Nutraceutical Examples

The nutraceuticals listed in Table 3 were incorporated into exemplary dispersions, which resulted in nanoparticle or complexed solids comprising the nutraceutical encapsulated or at least partially encapsulated by the organic cane sugar carrier. It was confirmed by mass spectrometry and/or NMR spectroscopy that, in general, the composition of the nutraceutical starting material was not altered upon creating the dispersion and recovering the composition from the dispersion.

TABLE 3
EXEMPLARY NUTRACEUTICALS

| Nutraceutical | Reported Benefits |
|---|---|
| Moringa | Anti-oxidant and anti-inflammatory |
| Vitamin D3 | Various |
| L-Theanine | Anti-anxiety |
| Methylsulfonylmethane | Anti-inflammatory |
| (MSM) | |
| Asian Ginseng | Performance |
| Guarana | Anti-oxidant and stimulant |
| Ginger Root | Gastroprotective, Anti-oxidant/anti- |
| | inflammatory, Neuroprotective, |
| | Cardioprotective |
| Ceylon Cinnamon | Various |
| Curcumin | Anti-inflammatory |
| Magnesium Oxide | Performance |
| Tumeric | Anti-inflammatory |
| Caralluma Fimbrita | Various |
| Beet Root | Various |
| Green Tea Extract | Various |
| Garcinia Cambogia | Various |
| Conjugated linoleic acid | Various |
| (CLA) | |
| CoQ10 | Anti-oxidant/anti-inflammatory |
| Maca | Various |
| Nutraceutical | Reported Benefits |
| Echinacea | Various |
| L-Tryptophan | Anti-anxiety |
| Coconut Oil | Various |
| Milk Thistle | Anti-oxidant |
| Lime | Various |
| Valerian Root | Anti-anxiety |
| St. John's Wort | Various |
| Kava Tincture | Various |
| Black seed oil | Various |
| Melatonin | Sleep aid |
| L-Carnitine | Various |
| L-Thyroxine | Various |
| Lemongrass | Various |
| Lemon | Various |
| Fennel | Various |
| Oregano | Various |
| Peppermint | Various |

### 3. Therapeutic Agent Examples

The therapeutic agents listed in Table 4 were incorporated into exemplary dispersions as indicated, which resulted in solid nanoparticles, or in some cases syrups, comprising the therapeutic agent encapsulated or at least partially encapsulated by the organic cane sugar carrier. It was confirmed by mass spectrometry and/or NMR spectroscopy that, in general, the composition of the therapeutic agent starting material was not altered upon creating the dispersion and recovering the composition from the dispersion.

A representative dispersion for preparing the therapeutic agent compositions (e.g., complexes, nanoparticles, or syrups) comprised 2 grams of therapeutic agent, about 55 grams of organic cane sugar, and about 38 grams of water. The resulting dispersion was heated to about 115° C. and optionally cooled/hardened/ground into powder.

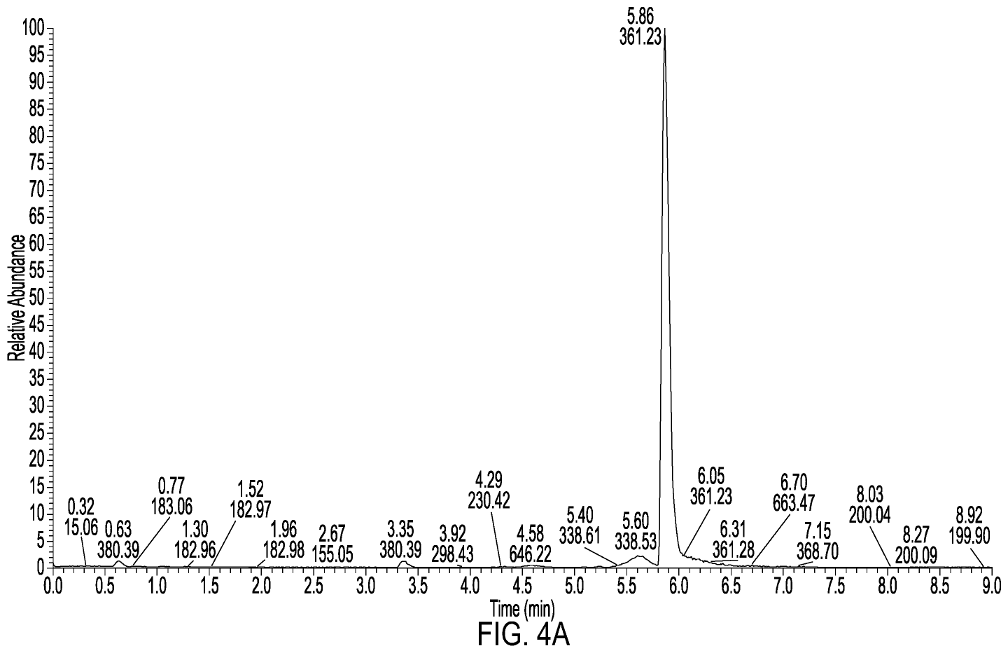

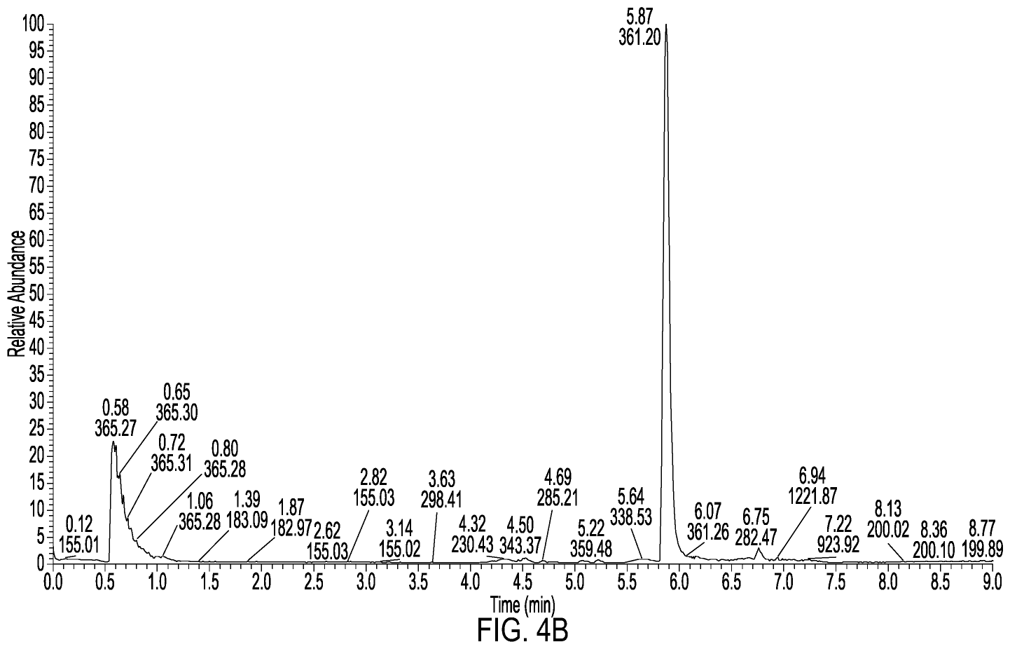

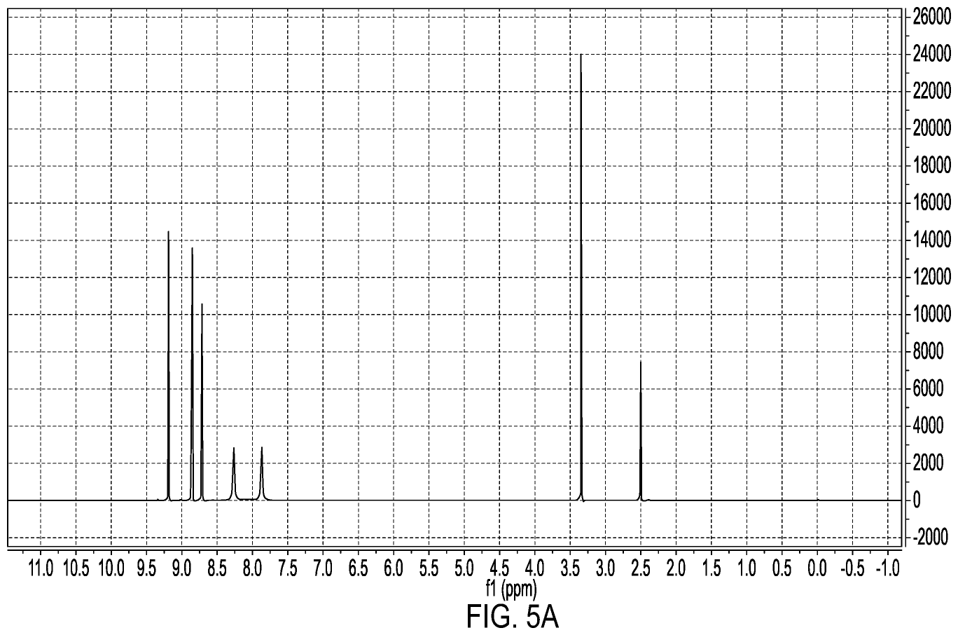

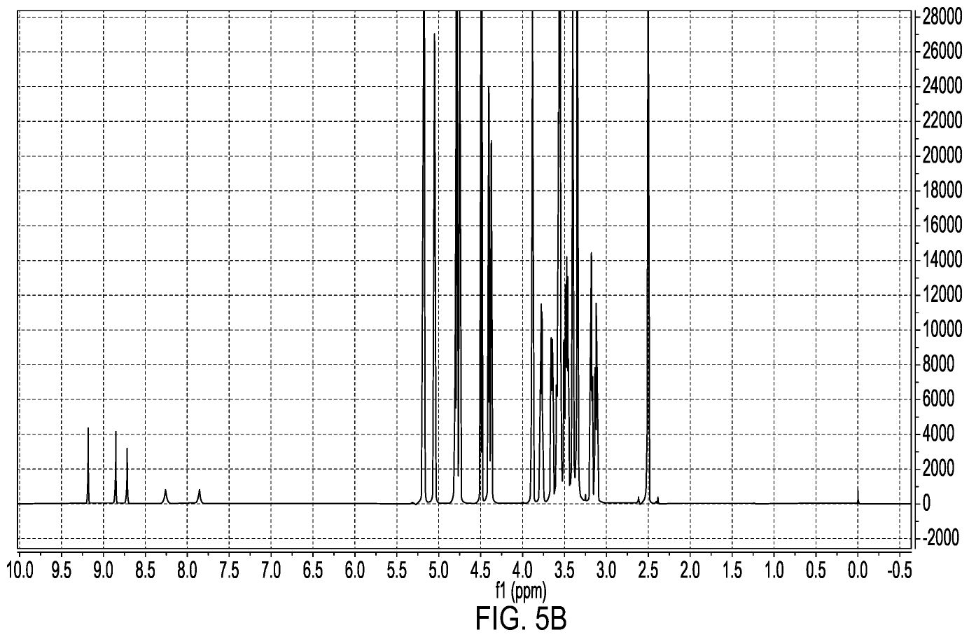

For many of the sample compositions below, the stability of the therapeutic agent after recovery from the dispersion was confirmed by mass spectrometry against a reference sample of therapeutic agent. A representative example can be seen in FIGS. 4A-B, which shows an encapsulated fenofibrate sample (FIG. 4B) compared to a reference standard (FIG. 4A), demonstrating no decomposition occurred during preparation of the dispersion and recovery process. NMR spectroscopy was used for a similar purpose (see FIGS. 5A-B).

Samples for mass spectrometry were weighed and then concentrated to 5 mg/mL. The samples were further diluted to 0.1 mg/mL for analysis in 50/50 water to methanol. Standards were prepared to 20 μg/mL in optima grade methanol or water. The following standards were prepared in water at 20 μg/mL: bethanechol chloride, chloroquine diphosphate, cyclobenzaprine hydrochloride, D-penicillamine, ondansetron hydrochloride, venlafaxine hydrochloride, and donepezil hydrochloride. The risperidone standard and batch were both prepared in methanol due to insolubility in water.

Mass spectrometry results revealed that therapeutic agents aside from those marked with an asterick (*) were observed to remain stable within detection limits after recovery from the dispersion.

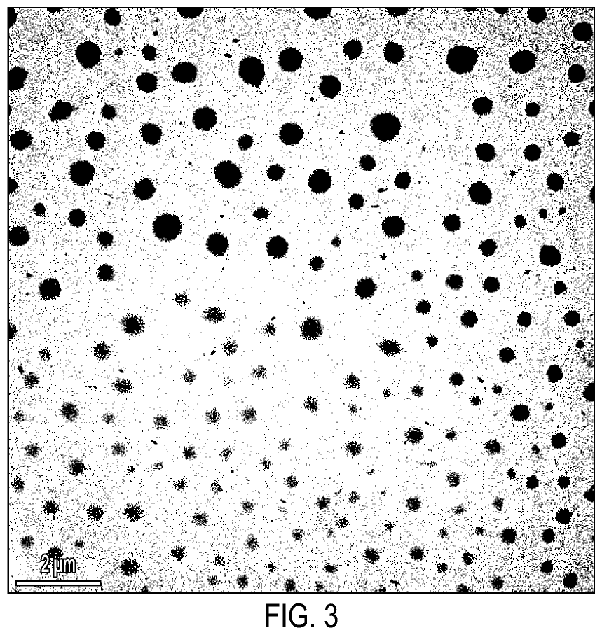

TABLE 4
EXEMPLARY THERAPEUTIC AGENTS

| Therapeutic | Melting | | |
|---|---|---|---|
| Agent | Temperature | LogP | Form/Outcome |
| Amiodarone* | 156-165° C. | 7.2 | Solid complex/nanoparticle |
| | | | composition recovered |
| Aripiprazole | 137-140° C. | 4.9 | Solid complex/nanoparticle |
| | | (predicted) | composition recovered |
| Aspirin | 134-136° C. | 1.18 | Solid complex/nanoparticle |
| | | | composition recovered |
| Atenolol | 147-160° C. | 0.16 | Solid complex/nanoparticle |
| | | | composition recovered |
| Bisacodyl | 138° C. | 3.61 | Solid complex/nanoparticle |
| | | (predicted) | composition recovered |
| Buspirone | 201-203° C. | 2.63 | Syrup recovered; syrup |
| | | | cooled, hardened, and milled |
| | | | to fine powder |
| Bethanechol | 187-190° C. | −4.1 | Solid complex/nanoparticle |
| chloride | | | composition recovered |
| Brompheniramine | 25° C. | 3.4 | Solid complex/nanoparticle |
| | (liquid | | composition recovered |
| | at room | | |
| | temperature), | | |
| | boiling | | |
| | temperature | | |
| | 147-152° C. | | |
| Carbamazepine | 189-190° C. | 2.77 | Solid complex/nanoparticle |
| | | | composition recovered |
| Citalopram | 156-157° C. | 3.76 | Solid complex/nanoparticle |
| | | | composition recovered |
| Clopidogrel | 158° C. | 4.03 | Solid complex/nanoparticle |
| | | (predicted) | composition recovered |
| Colchicine | 150-160° C. | 1.07 | Solid complex/nanoparticle |
| | | | composition recovered |
| Cyclobenzaprine | 217° C. | 5.2 | Solid complex/nanoparticle |
| hydrochloride | | | composition recovered |
| Dextro- | 111° C. | 3.49 | Solid complex/nanoparticle |
| methorphan | | (predicted) | composition recovered |
| D-penicillamine* | 202-204° C. | −1.78 | Solid complex/nanoparticle |
| | | | composition recovered |
| Chlorozoxazone | 191-192° C. | 1.6 | Solid complex/nanoparticle |
| | | | composition recovered |
| Donepezil* | 223-227° C. | 4.7 | Solid complex/nanoparticle |
| | | | composition recovered |
| Doxepin | 187-189° C. | 4.29 | Solid complex/nanoparticle |
| | | | composition recovered |
| Diazoxide | 330-331° C. | 1.20 | Solid complex/nanoparticle |
| | | | composition recovered |
| Diphenyl- | 295° C. | 2.47 | Solid complex/nanoparticle |
| hydantoin | | | composition recovered |
| Famotidine | 163-164° C. | −0.64 | Solid complex/nanoparticle |
| | | | composition recovered |
| Fluoxetine | 179-182° C. | 4.05 | Syrup recovered; syrup |
| Hydrochloride | | | cooled, hardened, and milled |
| | | | to fine powder |
| Fingolimod* | 102-107° C. | 5.5 | Solid complex/nanoparticle |
| | | | composition recovered |
| Glimepiride | 207° C. | 3.12 | Solid complex/nanoparticle |
| | | (predicted) | composition recovered |
| Glipizide | 208-209° C. | 1.91 | Solid complex/nanoparticle |
| | | | composition recovered |
| Guaifenesin | 78-79° C. | 1.39 | Solid complex/nanoparticle |
| | | | composition recovered |
| Hydro- | 274° C. | −0.07 | Solid complex/nanoparticle |
| chlorothiazide | | | composition recovered (see |
| | | | FIG. 3) |
| Ibuprofen | 75-77° C. | 3.97 | Solid complex/nanoparticle |
| | | | composition recovered |
| Imatinib* | 208-210° C. | 3.0 | — |
| Itraconazole | 170° C. | 5.66 | Solid complex/nanoparticle |
| | | | composition recovered |
| Lamotrigine | 216-218° C. | 1.93 | Solid complex/nanoparticle |
| | | | composition recovered |
| Lisinopril | 160° C. | −1.01 | Solid complex/nanoparticle |
| dihydrate | | | composition recovered |
| Loratadine | 134-136° C. | 4.55 | Solid complex/nanoparticle |
| | | (predicted) | composition recovered |
| Meloxicam | 254° C. | 0.1 | Solid complex/nanoparticle |
| | | | composition recovered |
| Mesalamine | 283° C. | −0.29 | Did not result in successful |
| | | | composition |
| Naproxen | 153° C. | 3.18 | Solid complex/nanoparticle |
| | | | composition recovered |
| Olanzapine | 195° C. | 4.09 | Solid complex/nanoparticle |
| | | | composition recovered |
| Pyridoxine | 318° C. | −0.77 | Solid complex/nanoparticle |
| | | | composition recovered |
| Pyrazinamide | 189-191° C. | −0.60 | Solid complex/nanoparticle |
| | | | composition recovered |
| Selegiline | 141-142° C. | 2.7 | Solid complex/nanoparticle |
| | | | composition recovered |
| Vancomycin | >190° C. | −3.1 | Solid complex/nanoparticle |
| | | | composition recovered |
| Amoxicillin | 194° C. | 0.87 | Solid complex/nanoparticle |
| | | | composition recovered |
| Doxycycline | 201° C. | 0.63 | Syrup recovered; syrup |
| | | | cooled, hardened, and milled |
| | | | to fine powder |
| Erythromycin | 135-140° C. | 2.6 | Solid complex/nanoparticle |
| | | | composition recovered |
| Fluconazole | 139° C. | 0.5 | Solid complex/nanoparticle |
| | | | composition recovered |
| Levodopa | 276-278° C. | 0.05 | Solid complex/nanoparticle |
| | | | composition recovered |
| Levofloxacin | 225-227° C. | 2.1 | Solid complex/nanoparticle |
| | | | composition recovered |
| Levothyroxine | 231-233° C. | 4.0 | Solid complex/nanoparticle |
| | | | composition recovered |
| Ondanestron | 179° C. | 2.4 | Solid complex/nanoparticle |
| | | | composition recovered |
| Prednisone | 233° C. | 1.46 | Solid complex/nanoparticle |
| | | | composition recovered |
| Sertraline | 243-249° C. | 5.15 | Syrup recovered; syrup |
| | | (predicted) | cooled, hardened, and |
| | | | milled to fine powder |
| Baclofen | 194-208° C. | 1.3 | Solid complex/nanoparticle |
| | | | composition recovered |
| Benztropine | 135° C. | 4.27 | Solid complex/nanoparticle |
| | | | composition recovered |
| Carvedilol | 238° C. | 3.8 | Solid complex/nanoparticle |
| | | | composition recovered |
| Cetirizine | 110-115° C. | 2.8 | — |
| Chloroquine | 289° C. | 4.63 | Solid complex/nanoparticle |
| | | | composition recovered |
| Ethinylestradiol | 182-184° C. | 3.67 | — |
| Fenofibrate | 233° C. | 5.2 | Solid complex/nanoparticle |
| | | | composition recovered |
| Insulin | 233° C. | | — |
| Irbesartan | 180-181° C. | 4.5 | Solid complex/nanoparticle |
| | | | composition recovered |
| Lovaststin | 152-175° C. | 4.08 | Solid complex/nanoparticle |
| | | | composition recovered |
| Memantine | 258° C. | 3.28 | Solid complex/nanoparticle |
| | | | composition recovered |
| Metronidazole | 159-161° C. | −0.02 | — |
| Nifedipine | 171-175° C. | 2.20 | Solid complex/nanoparticle |
| | | | composition recovered |
| Phenytoin | 295-298° C. | 2.47 | — |
| Piaglitazone | 183° C. | 3.33 | Solid complex/nanoparticle |
| | | (predicted) | composition recovered |
| Ranitidine | 133° C. | 0.2 | Solid complex/nanoparticle |
| | | | composition recovered |
| Risperidone | 170° C. | 2.63 | 0Solid complex/nanoparticle |
| | | (predicted) | composition recovered |
| Sildenafil | 189-190° C. | 1.9 | Solid complex/nanoparticle |
| | | | composition recovered |
| Spironolactone | 134-135° C. | 2.78 | Solid complex/nanoparticle |
| | | | composition recovered |
| Sulfamethoxazole | 169° C. | 0.89 | Solid complex/nanoparticle |
| | | | composition recovered |
| Trimethoprim | 199-203° C. | 0.91 | Solid complex/nanoparticle |
| | | | composition recovered |
| Venlafaxine | 217° C. | 2.74 | Solid complex/nanoparticle |
| hydrochloride | | (predicted) | composition recovered |
| Warfarin* | 151-161° C. | 2.70 | Solid complex/nanoparticle |
| | | | composition recovered |

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for making a therapeutic agent or nutraceutical composition, the method comprising:
a) forming a dispersion comprising:
i) a sugar, sugar alcohol, or sugar substitute;
ii) water; and
iii) an effective amount of the therapeutic agent or nutraceutical;
wherein the mass ratio of the therapeutic agent or nutraceutical to the sugar, sugar alcohol, or sugar substitute is from about 1:50 to 1:10; and the mass ratio of the sugar, sugar alcohol, or sugar substitute to the water is from about 1:8 to about 1:1.5;
b) heating the dispersion at a temperature equivalent to from 90° C. to 122° C. at a pressure of 1 atm for a sufficient time to reduce the volume of the dispersion; and
c) after the heating step, recovering the therapeutic agent or nutraceutical composition from the dispersion;
wherein the therapeutic agent or nutraceutical composition recovered from the dispersion is in a form selected from:
i) a solidified complex, or syrup, characterized by a physical aggregation or noncovalent chemical association of the therapeutic agent or nutraceutical and the sugar, sugar alcohol, or sugar substitute; wherein the syrup, when present, has a viscosity ranging from about 15 cp to about 25,000 cp; or
ii) nanoparticles comprising the therapeutic agent or nutraceutical encapsulated by the sugar, sugar alcohol, or sugar substitute, the nanoparticles having a particle size ranging from 200 nm to 2,000 nm;
wherein the therapeutic agent, when present, has a molecular weight of 120 g/mol to 6,000 g/mol; and when the therapeutic agent is a solid at a temperature equivalent to 25° C. at a pressure of 1 atm, the therapeutic agent has a melting point of 100° C. to 390° C.; and when the therapeutic agent is a liquid at a temperature equivalent to 25° C. at a pressure of 1 atm, the therapeutic agent has a boiling point of at least 130° C.

2. The method of claim 1, wherein the dispersion is heated at a temperature equivalent to from about 104° C. to about 116° C. at a pressure of 1 atm.

3. The method of claim 1, further comprising cooling the dispersion after the heating step.

4. The method of claim 1, wherein the mass ratio of the therapeutic agent to the sugar, sugar alcohol, or sugar substitute is about 1:20.

5. The method of claim 1, wherein the mass ratio of the sugar, sugar alcohol, or sugar substitute to the water is from about 1:6 to about 1:4 prior to the recovering step.

6. The method of claim 1, wherein the mass ratio of the sugar, sugar alcohol, or sugar substitute to the water is about 1:4 prior to the recovering step.

7. The method of claim 1, further comprising forming a powder of the therapeutic agent or nutraceutical composition after the recovering step.

8. The method of claim 1, wherein the therapeutic agent, when present, has a molecular weight of 120 g/mol to 1,000 g/mol.

9. The method of claim 1, wherein the therapeutic agent, when present, has a melting point of 130° C. to 390° C., when the therapeutic agent is a solid at a temperature equivalent to 25° C. at a pressure of 1 atm.

10. The method of claim 1, wherein the therapeutic agent, when present, has an octanol-water partition coefficient (logP) of from −5 to 8.

11. The method of claim 1, wherein wherein the therapeutic agent or nutraceutical composition is free of any cannabinoid.

12. The method of claim 1, wherein the sugar, when present, comprises allulose, glucose, dextrose, fructose, galactose, sucrose, lactose, maltose, trehalose, maltodextrin, or a combination thereof

13. The method of claim 1, wherein the sugar, when present, comprises cane sugar.

14. The method of claim 1, wherein the sugar alcohol, when present, comprises xylitol, erythritol, or a combination thereof

15. The method of claim 1, wherein the sugar substitute, when present, comprises sucralose.

16. A product comprising the therapeutic agent or nutraceutical composition prepared by the method of claim 1.

17. A therapeutic agent or nutraceutical composition consisting essentially of a sugar, sugar alcohol, or sugar substitute; an effective amount of the therapeutic agent or nutraceutical; wherein when the therapeutic agent or nutraceutical composition is in the form of a solid complex or nanoparticles, the therapeutic agent or nutraceutical composition comprises less than 25% water by weight of the composition; and when the therapeutic agent or nutraceutical composition is in the form of a syrup, the therapeutic agent or nutraceutical composition comprises 30% to 60% water by weight of the composition; wherein the therapeutic agent, when present, has a molecular weight of 120 g/mol to 6,000 g/mol; and when the therapeutic agent is a solid at a temperature equivalent to 25° C. at a pressure of 1 atm, the therapeutic agent has a melting point of 100° C. to 390° C.; and when the therapeutic agent is a liquid at a temperature equivalent to 25° C. at a pressure of 1 atm, the therapeutic agent has a boiling point of at least 130° C.

18. The therapeutic agent or nutraceutical composition of claim 17, wherein the therapeutic agent, when present, has a melting point of 130° C. to 390° C., when the therapeutic agent is a solid at a temperature equivalent to 25° C. at a pressure of 1 atm.

19. The therapeutic agent or nutraceutical composition of claim 17, which is free of any cannabinoid.

20. A method comprising administering to a subject the therapeutic agent or nutraceutical composition of claim 17.
